(19) United States
(12) Patent Application Publication
(10) Pub. No.: US 2016/0346444 A1
(43) Pub. Date: Dec. 1, 2016

(54) CELLULAR CONTROL AND TISSUE REGENERATION SYSTEMS AND METHODS

(71) Applicant: KCI Licensing, Inc., San Antonio, TX (US)

(72) Inventor: David S. Zamierowski, Overland Park, KS (US)

(21) Appl. No.: 15/232,612

(22) Filed: Aug. 9, 2016

Related U.S. Application Data
(63) Continuation-in-part of application No. 13/245,677, filed on Sep. 26, 2011, now Pat. No. 9,408,956.
(60) Provisional application No. 61/386,380, filed on Sep. 24, 2010.

(51) Int. Cl. A61M 1/00; A61M 37/00

(52) U.S. Cl. CPC A61M 1/0084; A61M 37/00; A61M 2205/04; A61M 2205/05; A61M 2202/097; A61M 2205/3303; A61M 2230/30; A61M 2230/50; A61F 13/00068

(57) ABSTRACT

A system for in-vivo and ex-vivo tissue regeneration and cellular control, manipulation and management includes a source of cell manipulating factors, which are administered to a therapy zone via active pressure-differential components including a pump and a controller, or pulse-waves generated passively. A plate comprising tissue or an inert, bio-compatible material is provided in the therapy zone in proximity to a fluid flow manifold and tissue scaffolding. An embodiment of a medical cellular factor control system and method includes an implanted bio-reactor and a force transducer configured for supplying one or more cell-manipulating factors to a therapy zone surrounding the bio-reactor. Optionally, a concave-curved, internal reflector can be implanted in proximity to the bio-reactor, the internal reflector being configured for amplifying pressure waves in the therapy zone.

### CROSS-REFERENCE TO RELATED APPLICATION

This application is a continuation-in-part of U.S. patent application Ser. No. 13/245,677, filed Sep. 26, 2011, which is now U.S. Pat. No. 9,408,956, issued Aug. 9, 2016, which claims priority in U.S. Provisional Patent Application Ser. No. 61/386,380, filed Sep. 24, 2010, both of which are incorporated herein by reference.

### BACKGROUND OF THE INVENTION

1. Field of the Invention

The present invention relates generally to tissue repair, regeneration and engineering, cellular management devices and methods, and in particular to internal implantable and external surface-mount tissue generative devices accommodating cellular manipulative influence factors, which collectively can be introduced into and applied to tissue generation zones.

2. Description of the Related Art

In the medical field, which is broadly defined to include medicine, dentistry, veterinary medicine, etc., tissue reconstruction, closure, healing and repair are important aspects of many medical procedures. Such broad intentions generally involve control and manipulation at the cellular level, including the application of various influence factors known to signal cells to grow, reproduce, migrate, align and otherwise respond positively. Applying properly indicated influence factors, including pharmacological, chemical, antimicrobial, electromagnetic force (EMF), pressure differential (negative and positive), bioengineered cells for seeding, thermal energy, acoustic energy (e.g., ultrasound), mechanical and other influence factors, has been shown to significantly improve patient outcomes across a wide range of medical conditions and treatment procedures.

The prior art includes technologies and methodologies for positively influencing cellular migration and regeneration. For example, the Zamierowski U.S. Pat. No. 4,969,880; No. 5,100,396; No. 5,261,893; No. 5,527,293; and No. 6,071,267 are incorporated herein by reference and disclose the use of pressure gradients, i.e., vacuum/negative and positive pressure, to effect wound closure and fluid drainage from wounds, including surgical incision sites. Such pressure gradients can be established by applying porous foam material either internally or externally to a wound, covering same with a permeable, semi-permeable, or impervious membrane, and connecting a suction vacuum source thereto. Fluid drawn from the patient is collected for disposal. Such fluid control methodologies have been shown to achieve significant improvements in patient outcomes. Another aspect of fluid management, postoperative and otherwise, relates to the application of fluids to wound sites for purposes of irrigation, infection control, pain control, growth factor application, etc. Wound drainage devices are also used to achieve fixation and immobility of the tissues, thus aiding healing and closure. This can be accomplished by both internal closed wound drainage and external vacuum devices. Fixation of tissues in apposition can also be achieved by bolus tie-over dressings (e.g., Stent dressings), taping, strapping and (contact) casting.

Cells can be subjected to physical forces and/or chemical signals in order to achieve desired endpoints or therapy goals. For example, mechano-transduction force signal characteristics are known to influence cell behavior. Tension, compression and shear mechanical forces can be applied to encourage tissue regeneration and wound closure. Still further, electro-magnetic force (EMF) is known to encourage cellular growth and closure.

Cellular movement or “migration” is an important aspect of healing. The present invention applies various forces and other influences to accomplish cell migration in order to achieve closure and healing. In order for a cell to accomplish repair of an injured tissue, it must “migrate” into the defect and replace the missing cells and/or their functions in the damaged tissue. The same property is required for tissue engineering schema. Cells must multiply and migrate into desired shapes, beds or scaffolding to create a desired engineered tissue result. The present invention addresses regenerating and repairing a wide range of tissue types in connection with a virtually unlimited range of medical treatment procedures and desired outcomes.

Heretofore, there has not been available a cellular control system and method with the advantages and features of the present invention, including the combination of inter-tissue devices with influence factors.

### SUMMARY OF THE INVENTION

In the practice of one aspect of the present invention, a medical device is provided for implanting in a tissue space wherein regeneration is indicated under one or more influence factors. The implantable device can include a plate providing a differentiating barrier for controlling pressure, fluid flow, cells and other influence factors as input and output to an in-situ therapy zone, which can be internal or external or both relative to the patient. The plate can be absorbable or non-absorbable and autologous or non-autologous. Tissue regeneration/healing/repair scaffolding provides an interface between the plate and a tissue contact layer and can facilitate tissue regeneration with a matrix composition. An external cell-manipulating factor interface comprises fluid-conveying tubing, pressure (positive and negative) application components and EMF connections with the therapy zone.

In another aspect of the present invention, a bio-reactor is configured for implantation within tissue to aid in tissue and cellular growth. In combination with the implanted bio-reactor, a force transducer is configured for supplying one or more cell-manipulating factors or forces to a therapy zone surrounding the bio-reactor. Optionally, a concave-shaped reflector can be implanted in proximity to the bio-reactor to amplify pressure waves in the therapy zone.

### BRIEF DESCRIPTION OF THE DRAWINGS

FIG. 1 is a schematic diagram of a cellular control system embodying an aspect of the present invention.

FIG. 2 is a perspective view of an inter-tissue application of the cellular control system, including a fluid/pressure interface subsystem and an endotube.

FIG. 3 shows an alternative aspect including a cover adapted for rolling or furling on an access line or conduit.

FIG. 3A shows a conduit of the cellular control system extending through an incision in the skin surface.

FIG. 4 shows an implanted plate and a conduit position for placing a furled cover.

FIG. 5 shows the cover extending over a therapy zone.

FIG. 6 is a cross-sectional view thereof taken generally along line 6-6 in FIG. 5.

FIG. 7 shows another alternative aspect including fluid/pressure inlet and outlet conduits with manifolds engaging the plate.

FIG. 8 shows a flexible barrier film furled on a conduit and in position for extending over the plate.

FIG. 9 shows the flexible barrier film extending over the plate.

FIG. 10 shows the therapy zone closed by a tissue overlay.

FIG. 11 is a cross-sectional view taken generally along line 11-11 and FIG. 10.

FIG. 12 shows another alternative aspect including scaffolding installed with an endotube.

FIG. 13 shows an absorbable fabric hemostatic layer being applied over the scaffolding via the endotube.

FIG. 14 shows the completed assembly of the system in the therapy zone.

FIG. 15 shows the therapy zone covered by a tissue trapdoor plate.

FIG. 16 shows another alternative aspect of the present invention with inflow/outflow conduits extending into the therapy zone.

FIG. 17 is a cross-sectional view taken generally along line 17-17 in FIG. 16.

FIG. 18 shows another alternative aspect of the present invention with scaffolding located in the therapy zone including couplings.

FIG. 19 shows another aspect of the invention with multiple bellows-type pumps or pillars in the therapy zone.

FIG. 20 shows another aspect of the invention with a closed-loop endotube assembly in the therapy zone.

FIG. 21 is a cross-sectional view taken generally along line 21-21 in FIG. 20.

FIG. 22 is a schematic diagram similar to FIG. 1 showing another tissue regeneration and cellular control system embodying an alternative aspect of the present invention.

FIG. 23 shows a cross-sectional, elevational view of a bio-reactor engineering or wound (BREW) treatment system embodying an aspect of the present invention.

FIG. 24 shows a schematic diagram of a BREW treatment system embodying an aspect of the present invention.

FIG. 25 is a cross-sectional, elevational view of another embodiment of a BREW treatment system embodying an aspect of the present invention.

FIG. 26 shows a cross-sectional, elevational view of a BREW treatment system which embodies an additional aspect of the present invention.

FIG. 27 is a cross-sectional, elevational view of a BREW treatment system including a bio-reactor, a negative pressure surface device, and a second force transducer, the system embodying an aspect of the present invention.

FIG. 28 is a cross-sectional, elevational view of an alternative embodiment of a BREW treatment system embodying the present invention.

FIG. 29 shows a cross-sectional, elevational view of a BREW treatment system for use in wound treatment embodying the present invention.

FIG. 30 shows a cross-sectional, elevational view of a BREW treatment system including a concave-shaped internal reflector which embodies the present invention.

FIG. 31 is a diagram of a pressure wave transmission in a medium.

FIG. 32 is a diagram of opposing pressure waves in a medium, which can be created by an impact(s) on a media containment, such as a living organism or vessel. An opposing pressure wave can also be reflected by the containment.

FIG. 33a shows synchronized pressure waves in a medium, which result in a reinforced, standing pressure wave.

FIG. 33b shows a reinforced, standing pressure wave produced by synchronized pressure waves in a medium.

FIG. 33c shows a reflected pressure wave condition with increased frequency and shorter wavelength.

### DETAILED DESCRIPTION OF THE PREFERRED EMBODIMENTS

### I. Introduction and Environment

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Certain terminology will be used in the following description for convenience in reference only and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the embodiment being described and designated parts thereof. The words “horizontal” and “vertical” generally mean side-to-side and top-to-bottom, respectively. Said terminology will include the words specifically mentioned, derivatives thereof and words of a similar import.

Referring to the drawings in more detail, the reference numeral 2 generally designates a medical cellular control or tissue regeneration system embodying an aspect of the present invention. A primary intention of the cellular control system and method disclosed herein is tissue regeneration, which is broadly used to include tissue engineering, organ construction and tissue culture manufacturing. For example and without limitation on the generality of useful applications of the control system 2, a primary application disclosed herein is for controlling cellular regeneration and closure in an inter-tissue or intra-tissue space 4, which can be generally located between a contact layer 6 and an in-situ tissue surface 8, and is generally referred to as a “therapy zone.” The therapy zone 4 can be located at various treatment sites in or on a patient, although typically it will be at a pathology location which is the object of a medical procedure involving cellular manipulation by one or more of the factors identified at 12, including mechano/transductive, electro-magnetic force (EMF), pharmacological, chemical/antimicrobial, fluidic, bioengineered cells for seeding, thermal energy, acoustic energy (e.g., ultrasound), osmotic, oncotic, fluid pressure differential and others.

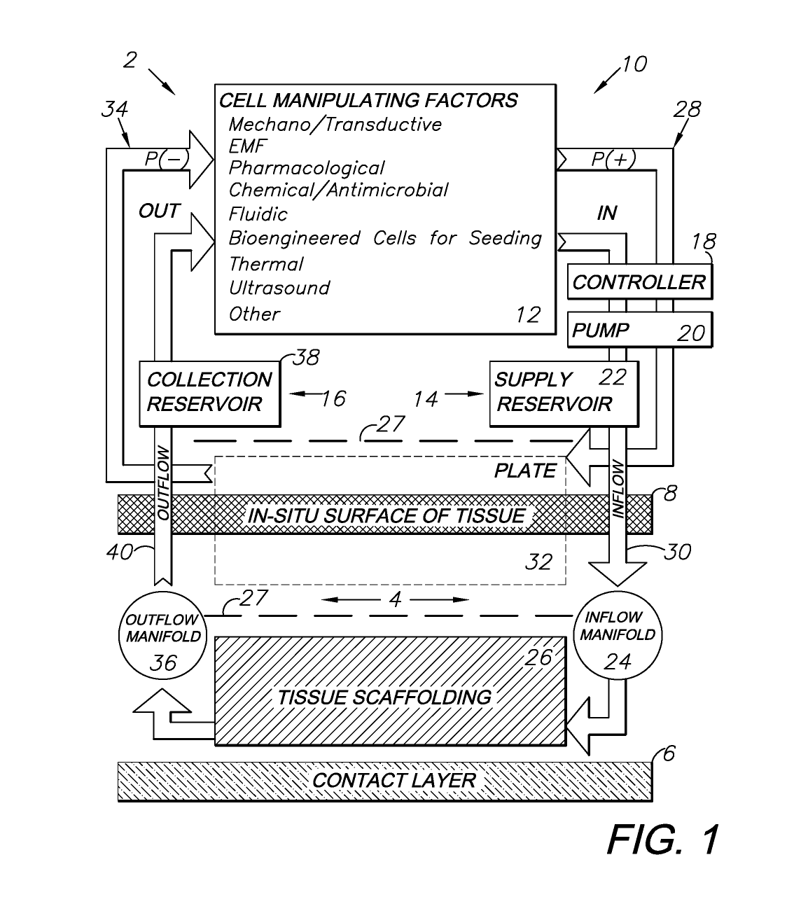

FIG. 1 shows a general interface 10 for applying the factors 12 to the therapy zone 4. The interface 10 includes a supply or inlet side 14 and an outlet side 16. By way of example and without limitation, the inlet side 14 can include a preprogrammed, digital controller 18 connected to and controlling a pump 20, which delivers the contents of a supply reservoir 22 to an inflow manifold 24 for application to tissue regeneration/healing/repair scaffolding 26. A suitable inlet conduit subsystem 28 is provided for delivering factors 12 via the inlet side 14. The inlet side 14 also includes a positive pressure conduit 30, which can be connected to a plate structure 32 in a plate area 27 of the therapy zone 4 via the controller 18 and the pump 20. Fluid flow in the plate area 27 can be influenced and directed by the plate structure 32.

An outlet side 16 of the interface 10 includes an outlet conduit subsystem 34 connected to an outflow manifold 36 from the scaffolding 26 and discharging to a collection reservoir 38. A negative pressure (NP) pressure conduit 40 connects the plate structure 32 to the factors 12, which can include a negative pressure source. For example, one or more pumps 20 can be located on either or both sides of the plate structure 32.

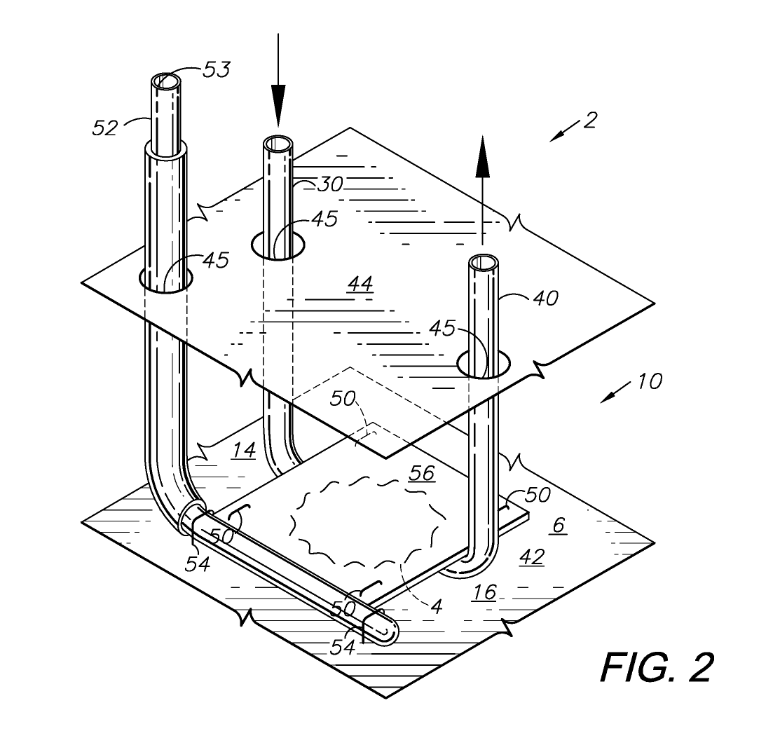

FIG. 2 shows a general configuration for the system 2 including a tissue bed 42 forming the tissue contact layer 6 and located below a skin surface 44. The inflow and outflow sides 14, 16 of the interface 10 can include respective inflow and outflow conduits 30, 40 extending through openings 45 in the skin surface 44 under the scaffolding 26 to the therapy zone 4. The scaffolding 26 can be retained in place on the tissue bed 42 by suitable anchors, such as scaffolding anchor clips 50, which can comprise staples, sutures or other suitable in-situ fasteners. An endotube 52 also extends through a skin surface opening 45 and is secured in place by endotube fasteners 54 (staples are shown) adjacent to scaffolding 56 located over the therapy zone 4. The endotube 52 is adapted for serving multiple functions, including placing and anchoring the scaffolding 56, and introducing multiple factors 12 into the therapy zone 4 via a lumen 53.

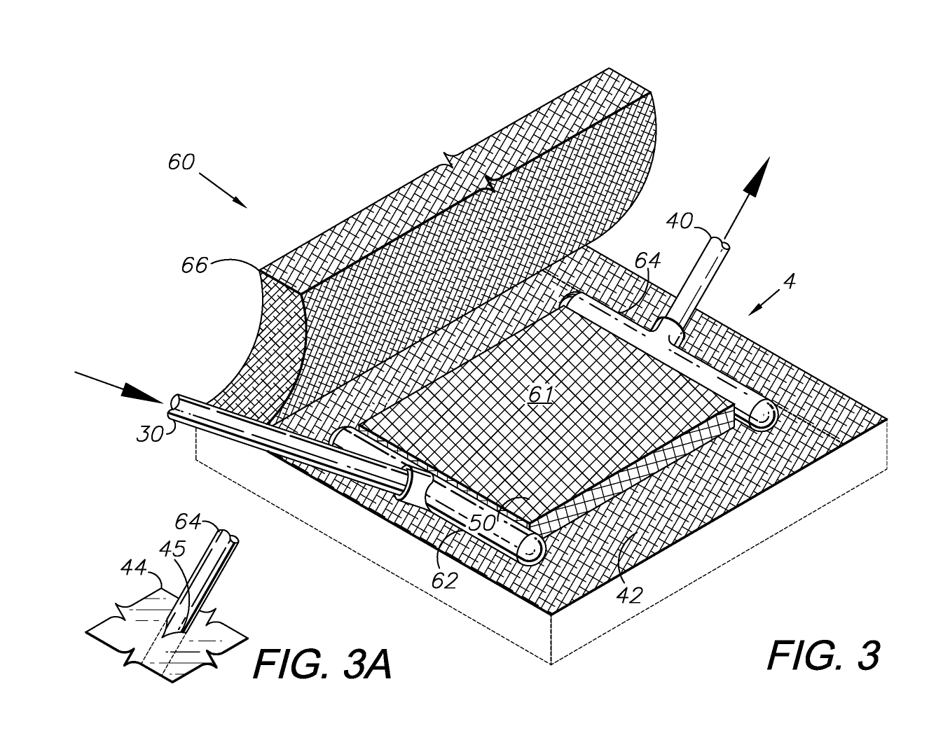

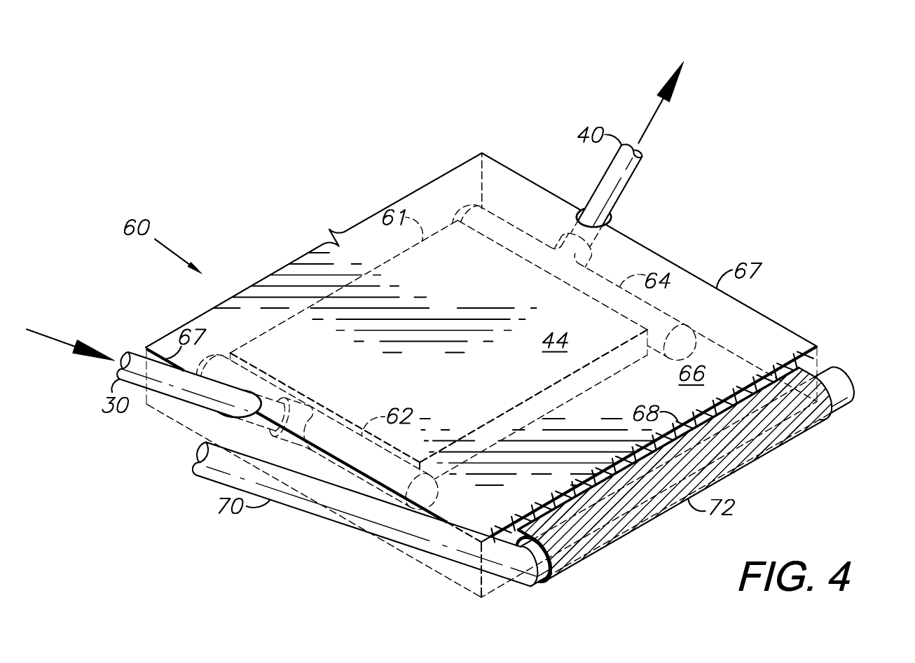

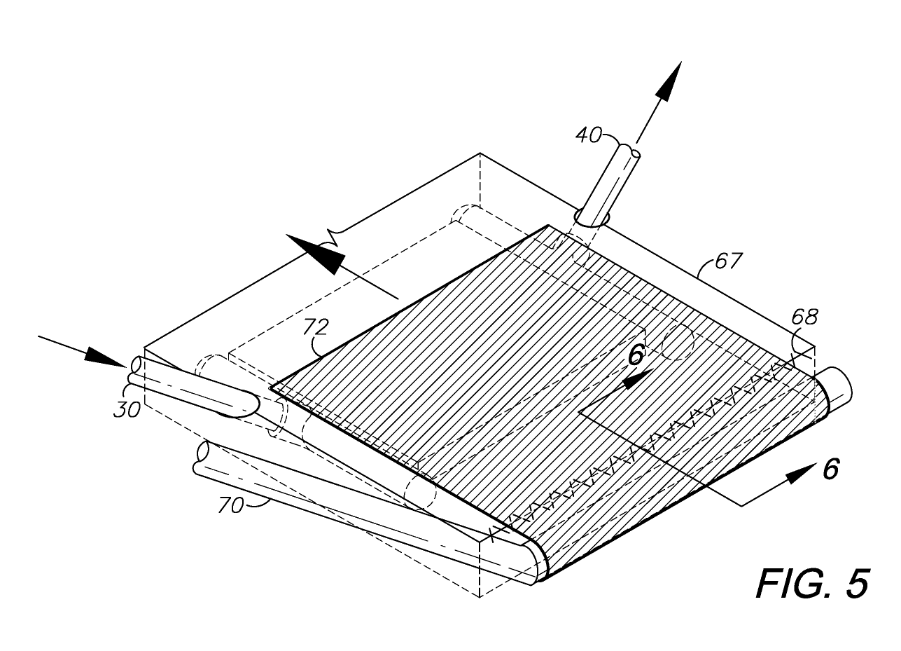

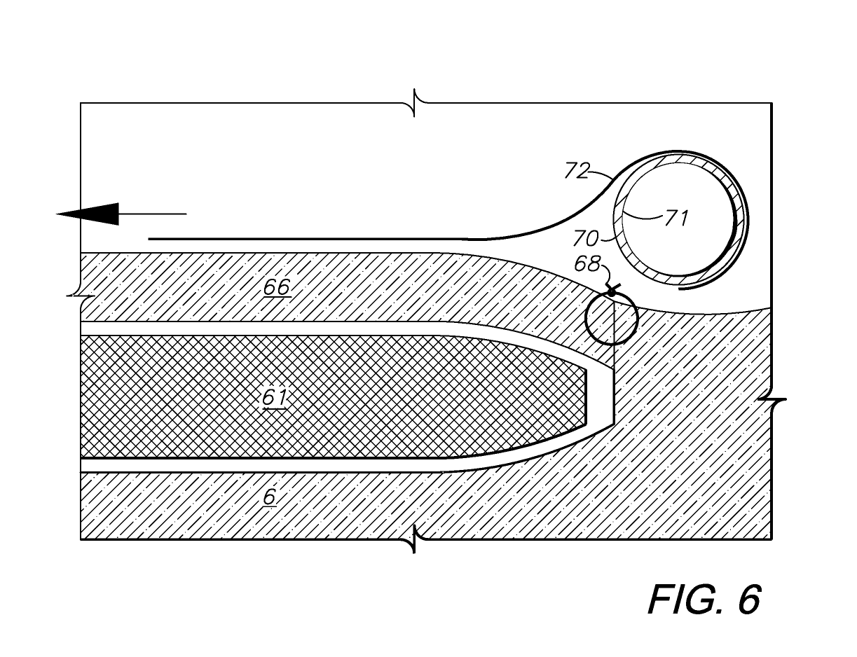

FIG. 3 shows a cellular control system 60 comprising another aspect of the invention with scaffolding 61 secured to the tissue bed 42 by the scaffolding fasteners 50 and positioned between inflow and outflow manifolds 62, 64, which are connected to inflow and outflow conduits 30, 40. The manifolds 62, 64 can be perforated, porous, semi-permeable or otherwise configured for communicating factors 12 with the scaffolding 61. A tissue flap or trapdoor plate 66 can be surgically opened by the incision 67 for access to the therapy zone 4 and closed as shown in FIG. 4 with a suture line 68 with the conduits 30, 40 extending through the flap incision lines 67 on either side of the tissue flap plate 66. A furled cover 72 is wrapped around an endotube 70 with an endotube bore 71 for placement in the therapy zone 4 and can be extended to a covering position generally over the scaffolding 61 (FIG. 5). As shown in FIG. 6, the cover 72 is adapted for covering the suture line 68 during healing and can comprise various suitable wound-dressing materials, including membranes and bio-absorbable dressings.

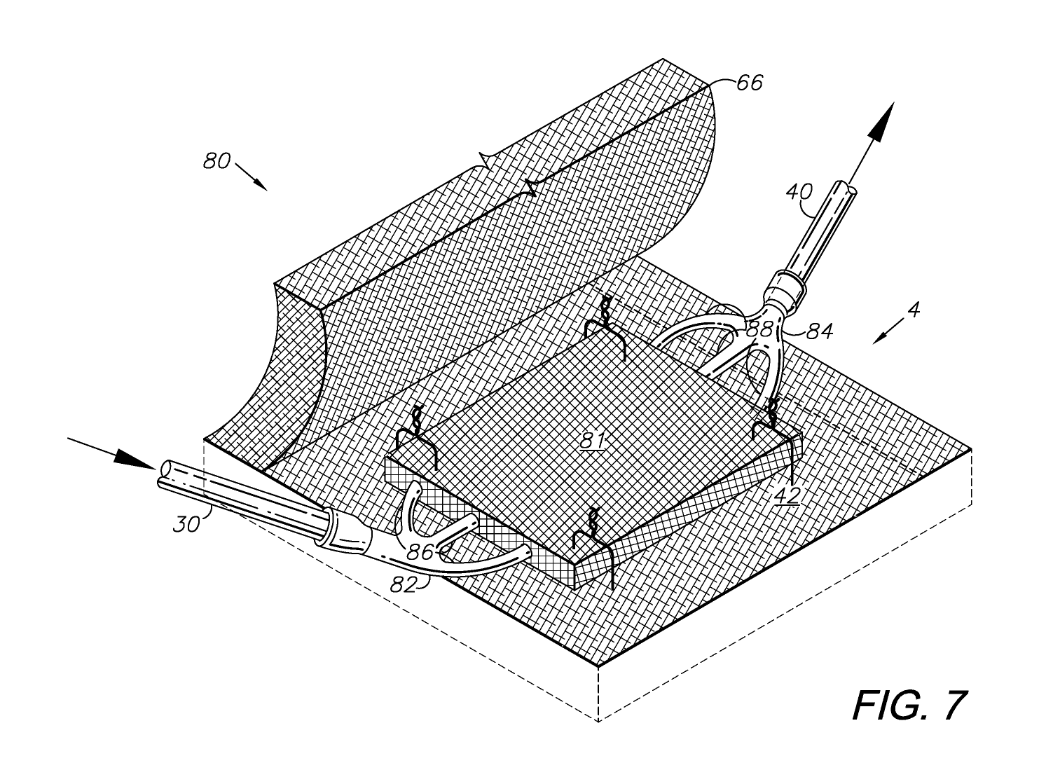

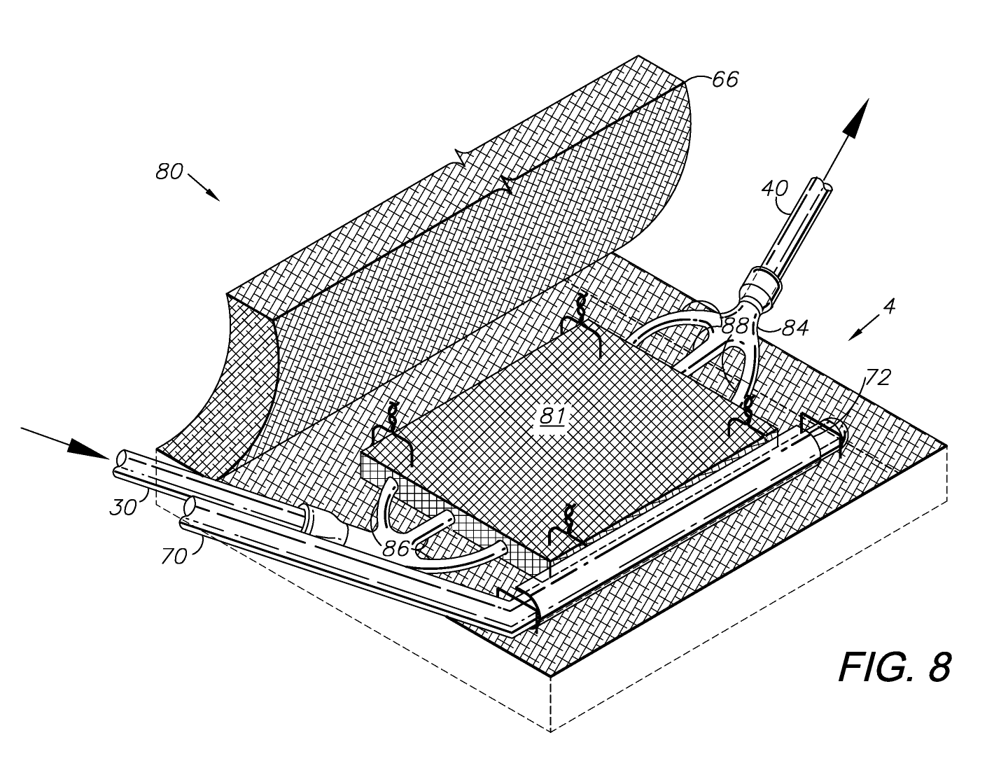

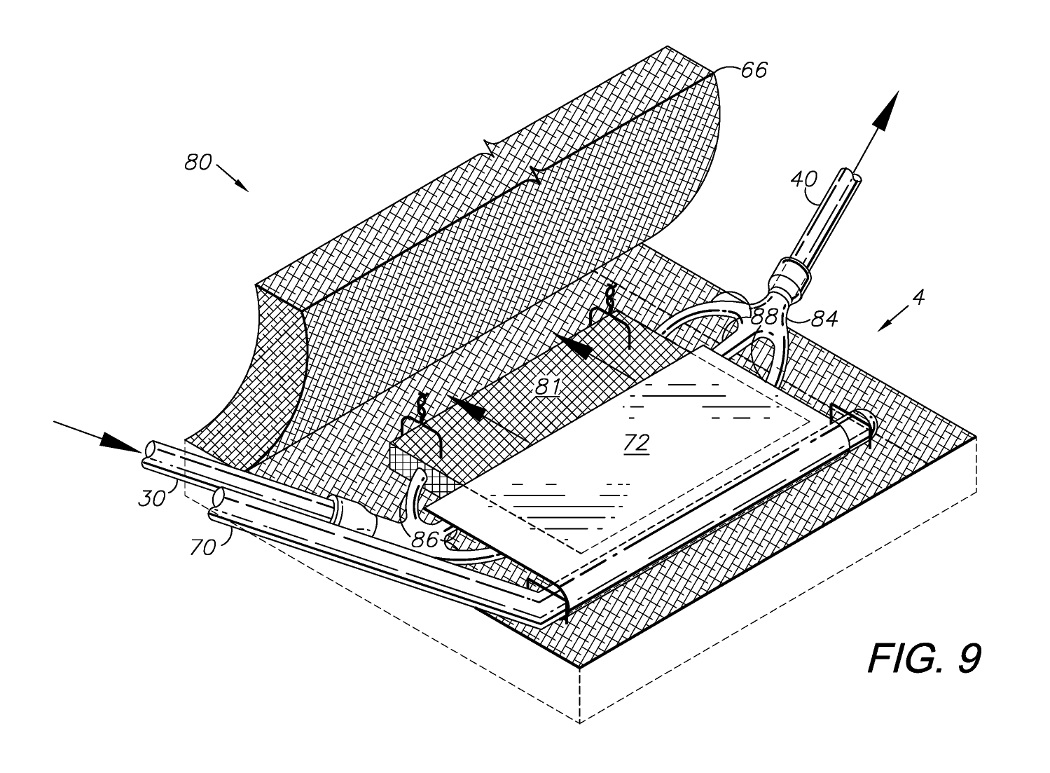

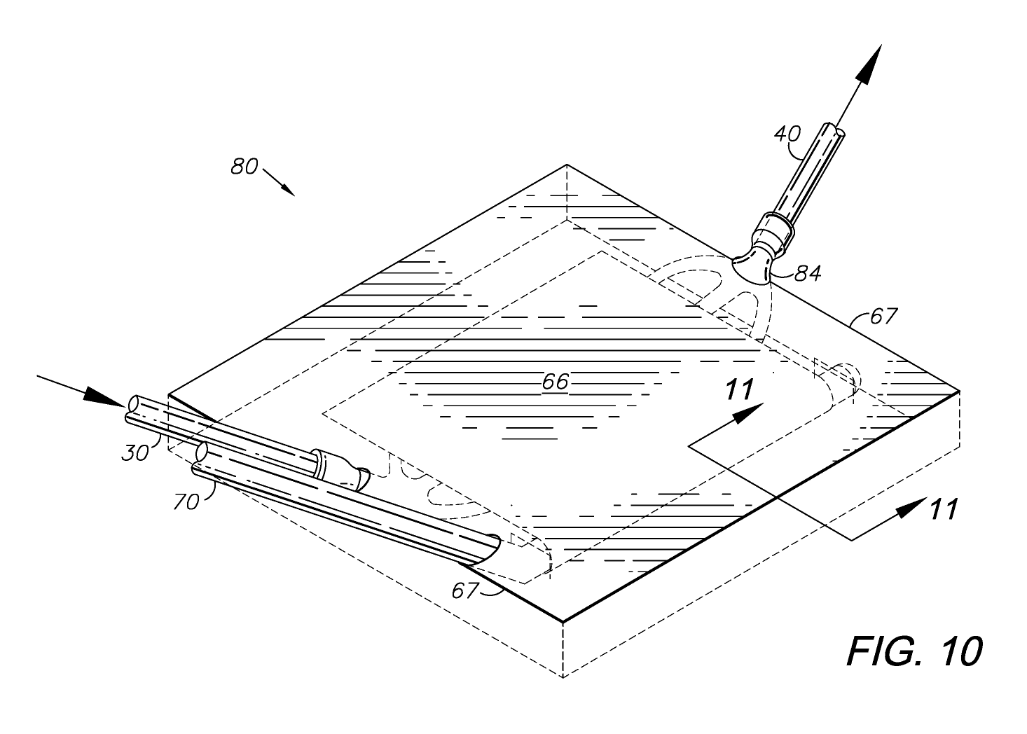

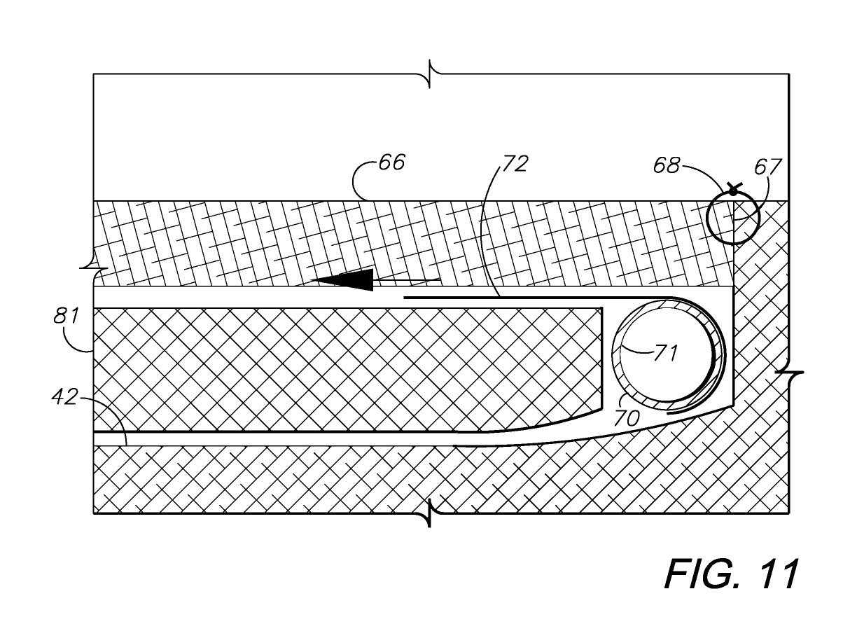

FIGS. 7-11 show another aspect of the invention comprising a cellular control system 80 with a fluid transfer element 81 inflow and outflow manifolds 82, 84 connected to conduits 30, 40 respectively and including respective manifold branches 86, 88 penetrating scaffolding for communicating fluids, pressure and other factors 12. The fluid transfer element 81 can comprise open-cell foam or some other suitable fluid-transferring material. As shown in FIGS. 8, 9 and 10, an endotube 70 with a furled cover 72 can be placed within the therapy zone 4 and covered by the tissue flap 66 whereby the cellular control system 60 is substantially contained within the enclosed therapy zone 4. Within such a closed environment, the cover 72 can be unfurled and extended by rotating the endotube 70 (FIG. 11).

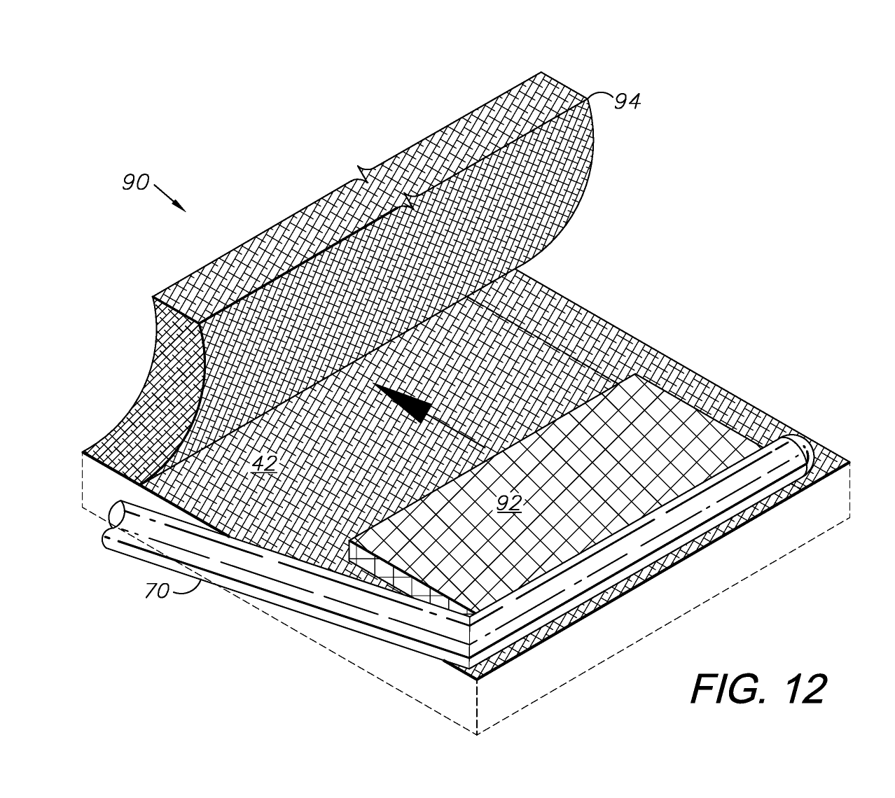

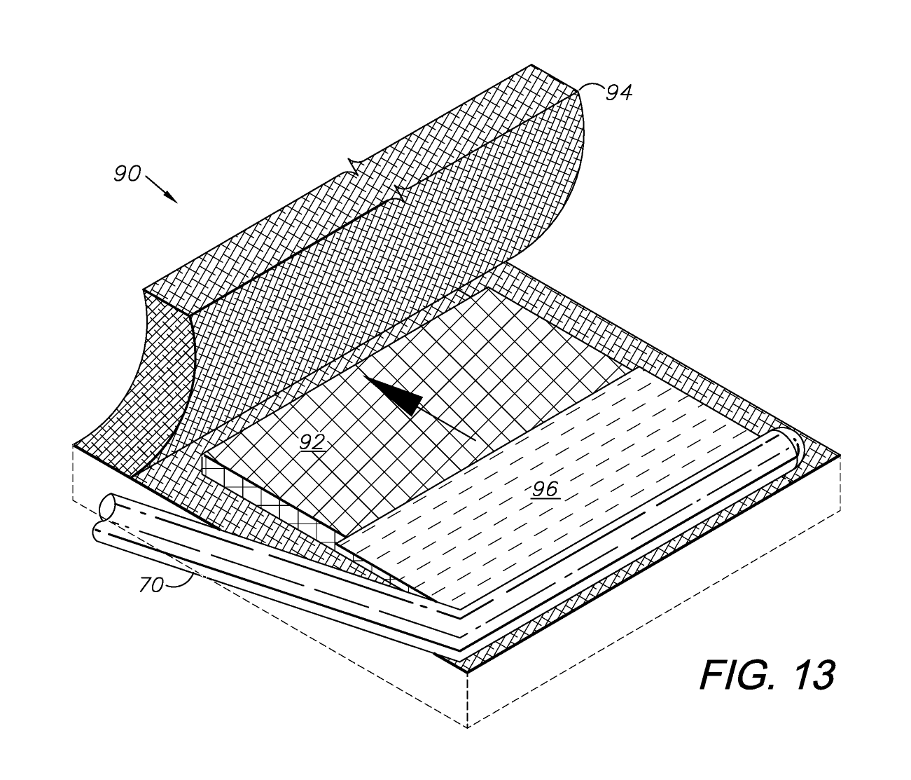

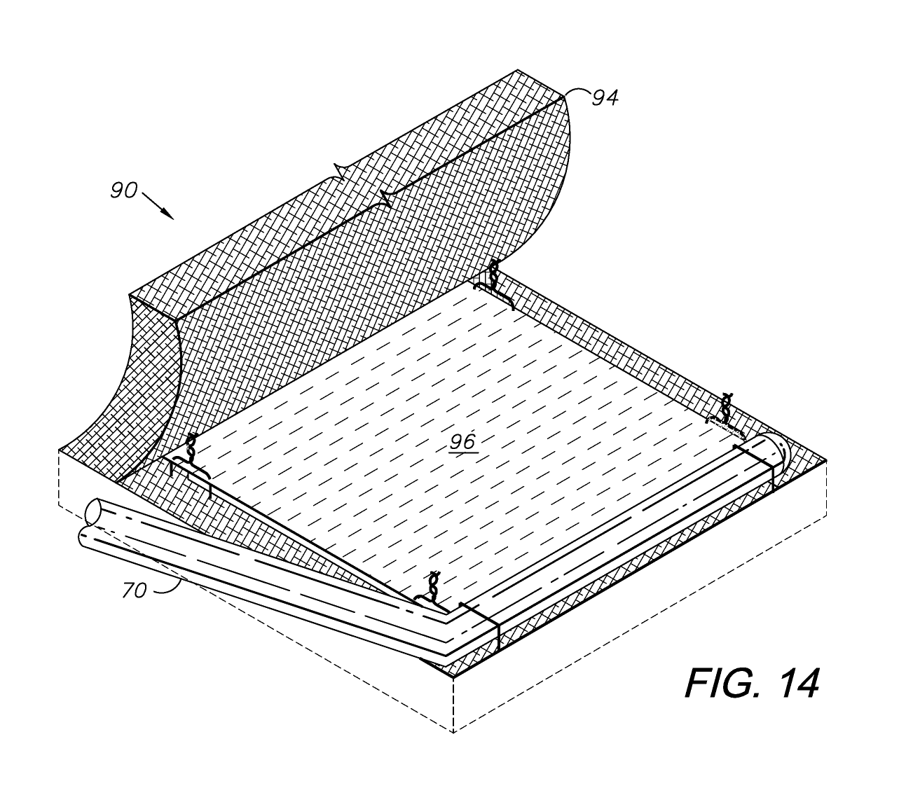

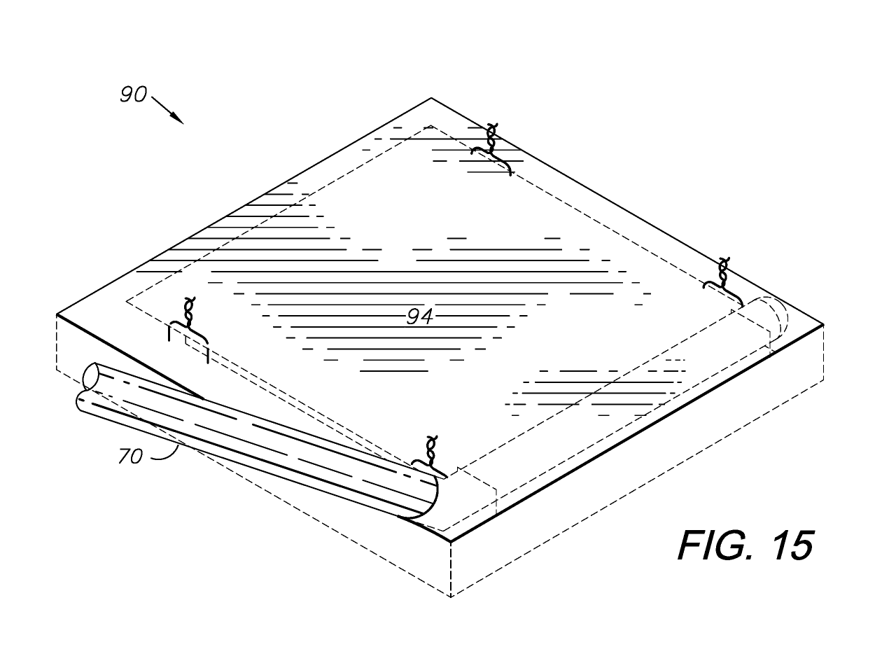

FIGS. 12-17 show a cellular control system 90 comprising another aspect of the invention and including scaffolding 92 adapted for placement in the therapy zone 4 on the tissue bed 6, which can be surgically exposed by lifting a tissue flap plate or trapdoor 94. As shown in FIG. 12, the scaffolding 92 can be placed with the endotube 52, which is positioned in the therapy zone 4 and in turn positions the scaffolding 92 over the tissue bed 6. An absorbable fabric hemostatic layer 96 is extended over the scaffolding 92 as shown in FIG. 13 and is secured to the tissue bed 6 with suitable fasteners 50, such as sutures or staples. The trapdoor 94 functions as the plate in this configuration and is placed over the scaffolding 92, the endotube 52 and the fabric hemostatic layer 96, as shown in FIG. 15. The tissue flap trapdoor plate 94 can be sutured in place over the therapy zone 4.

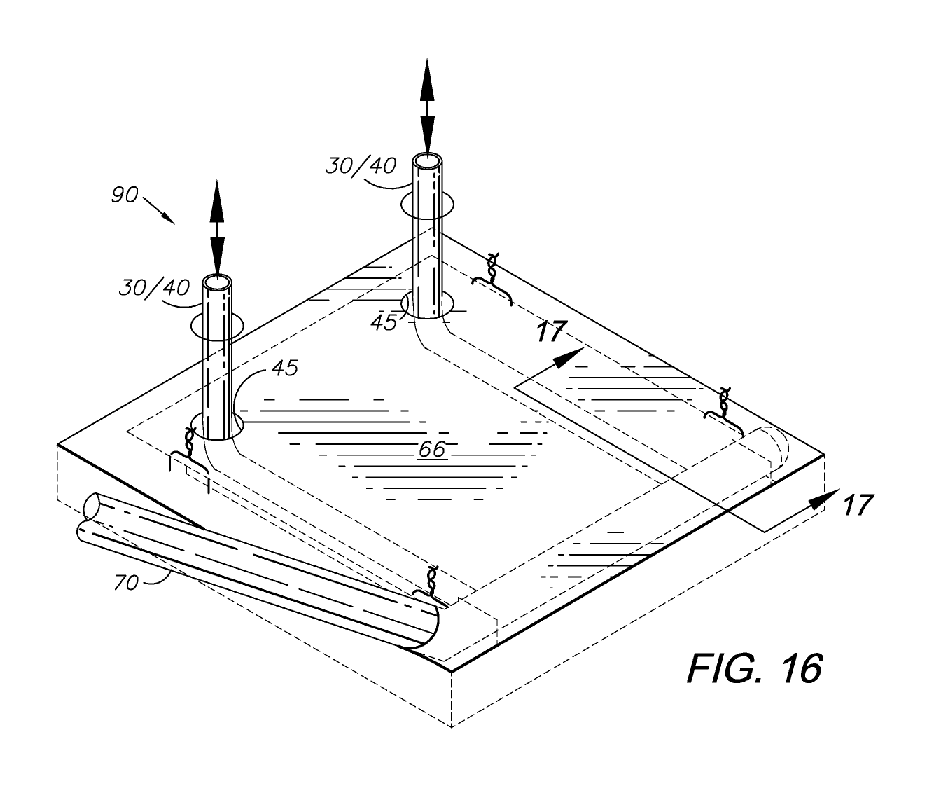

Inflow and outflow conduits 30, 40 are inserted through openings 45 in the tissue flap plate 94 as shown in FIG. 16 and can underlie the scaffold 94. Alternatively, the flow conduits 30, 40 can be placed before the scaffolding 92 is placed. The tissue flap plate 94 can be formed in subcutaneous tissue, with the flow conduits 46, 48 extending through skin surface openings 98 and penetrating to an appropriate depth to reach the therapy zone 4. Alternatively, in a surface application the tissue flap plate 94 can comprise the dermal and epidermal layers.

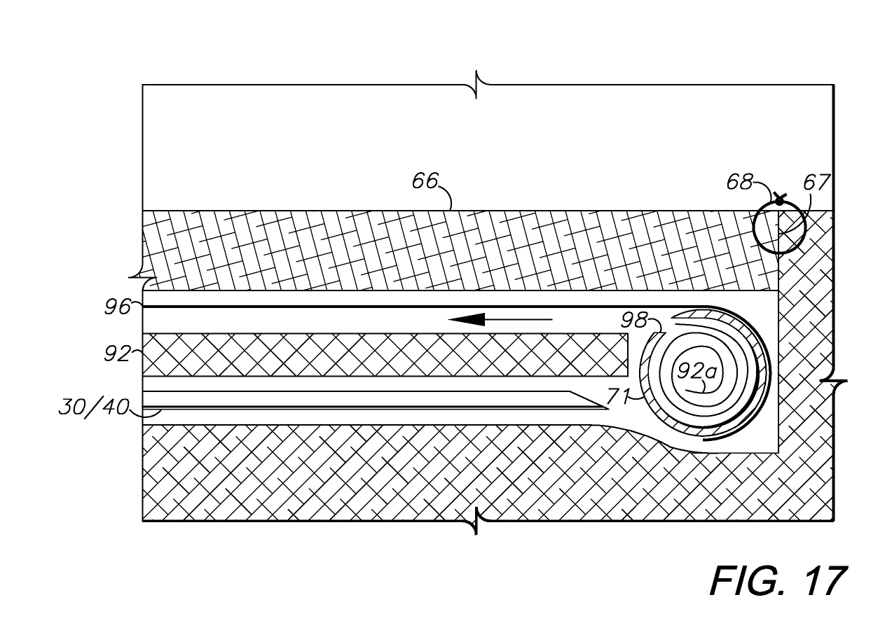

As shown in FIG. 17, the hemostatic fabric layer 96 can be wrapped around the endotube 52 for placement over the scaffolding 92. The endotube 52 can be slotted at 98 for accessing the lumen 53, which can receive the scaffolding 92 in a compression-rolled configuration 92a for unrolling into the therapy zone 4, for example, by a flexible rod extending through the endotube 52 for twisting externally to the patient.

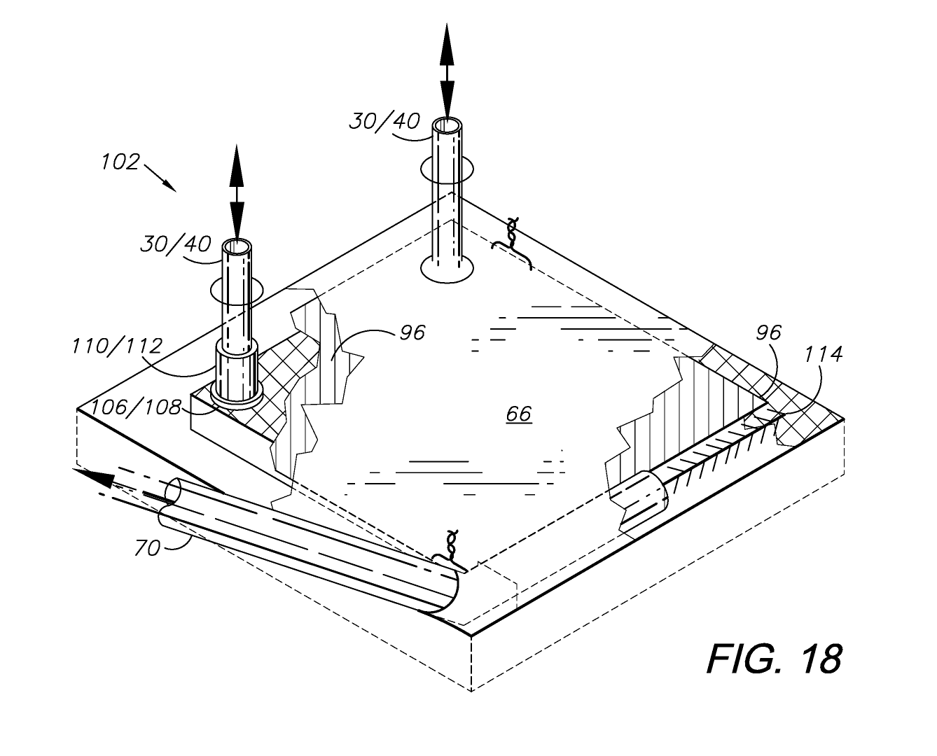

FIG. 18 shows a cellular control system 102 comprising another modified aspect of the invention and including scaffolding 104 with inflow and outflow female couplings 106, 108, which connect to the inflow and outflow conduits 30, 40 respectively via male couplings 110, 112. A barbed-strand, self-anchoring surgical suture 114 is shown being extended into the therapy zone 4 from the endotube 52. Such sutures are available from Quill Medical, Inc. of Research Triangle Park, North Carolina. See, for example, U.S. Pat. No. 7,056,331, which is incorporated herein by reference. The endotube 52 facilitates inserting the barbed suture 114 and “setting” its prongs by tugging on the outer end extending from the endotube 52 external to the patient for self-anchoring the suture 114.

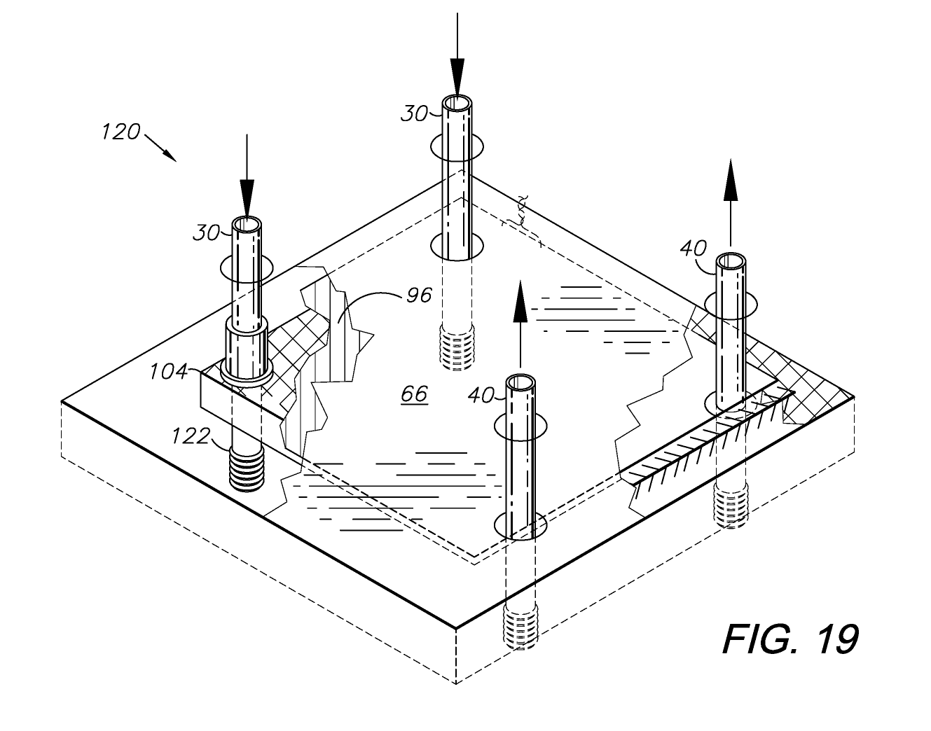

FIG. 19 shows a cellular control system 120 comprising another modified aspect of the present invention and including multiple bellows-action pillars 122 located below the scaffold 104 and fluidly connected to the inflow and outflow conduits 30, 40 respectively. The pillars 122 can reciprocally compress and expand in response to various pressures associated with the therapy zone 4. Such pressures can be externally-generated, e.g., by one or more of the factors 12, or internal pressures generated by the patient. Such pillars 122 can facilitate a “pumping” action with the cellular control system 120 by alternately expanding and contracting in order to move fluid into and out of the therapy zone 4.

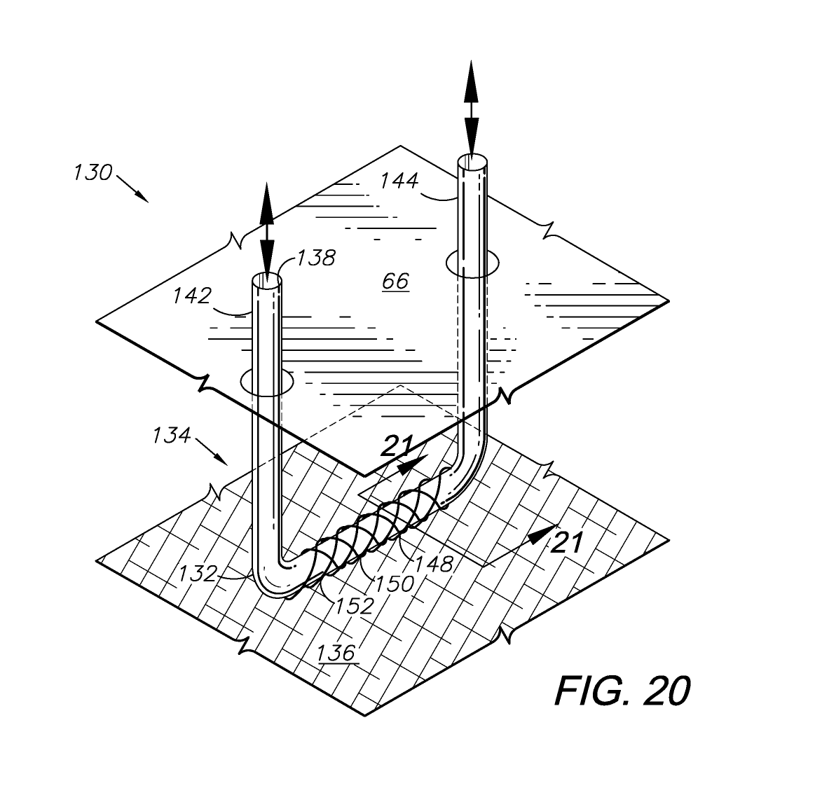

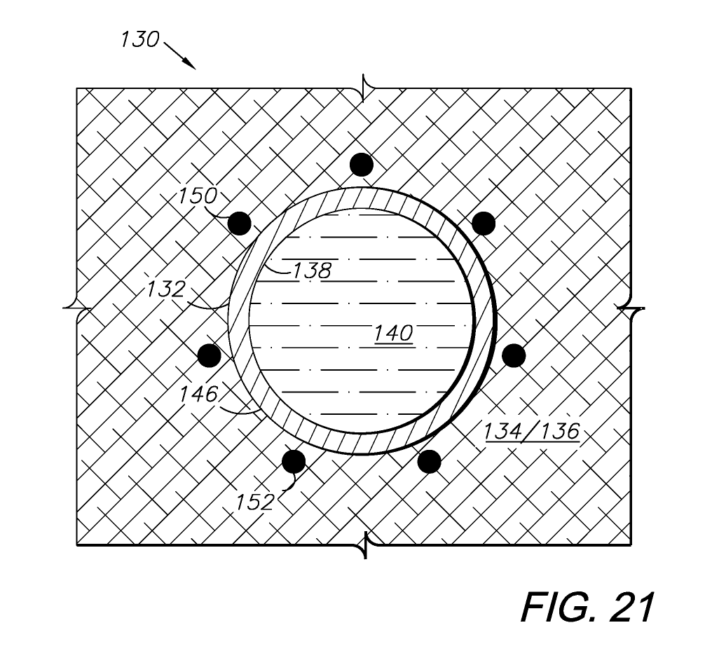

FIGS. 20 and 21 show a cellular control system 130 with a continuous loop endotube 132 forming the scaffolding 26 within a therapy zone 134 generally formed along the path of the endotube 132 through tissue 136. The endotube 132 includes a lumen 138, which can function as a conduit for introducing pharmacological and other substances 140, and/or extracting fluid from the patient. For example, the endotube 132 can be preloaded with cells for seeding the therapy zone 134. The endotube 132 forms inflow and outflow conduits 142, 144 with interchangeable functions. The endotube 132 includes an outer contact surface 146, which is adapted for engaging the tissue 136. The endotube 132 can be bioabsorbable, permanently implanted or extracted after completing a procedure. Moreover, the endotube 132 can be fabricated from a wide range of suitable materials chosen for compatibility with the therapeutic objectives of particular procedures. For example, semi-permeable materials can form pressure differentials and selectively transfer fluids. The endotube 132 can be perforated or slotted for fluid collection or dispersal. The external conduits 142, 144 can be connected to negative and/or positive pressure sources external to the therapy zone 134. Placement of the endotube 132 can be accomplished with a trocar instrument, by surgical incision or placement under a tissue flap or trapdoor 66.

An open mesh 148 comprising a matrix of threads or capillary-type tubes 150 forms a cellular control sleeve 152 over an endotube outer contact surface 146. The mesh 148 can introduce cells, facilitate cellular ingrowth, channel fluid evacuation, enhance tissue contact interaction and otherwise facilitate the treatment objectives. The range of suitable materials includes bioabsorbable materials, pharmacological release materials (e.g., antibiotics, growth factors, antiseptics, imaging materials and other suitable materials) and hollow tubes for communicating fluids. The mesh 148 can be extracted with the endotube 132, or left in place after extraction. Still further, the mesh 148 can comprise closure members, such as the barbed suture strands 114 available from Quill Medical, Inc., which are described above.

The tubular or thread configuration shown in FIGS. 20 and 21 includes the system and method embodiments described above, with their components formed in tubular shapes. These embodiments can include conduit size components (cm to mm range diameters), capillary size (mm range diameters) and nano size (micron diameters). Length can generally be any suitable length. The endotubes 132 can be fabricated and installed in various configurations, including straight, linearly-connected (series), parallel configurations, spiral, coil, circular, wave-like, etc. with the intention of optimizing recipient tissue bed positioning and ease of installation. Installation can be accomplished manually by palpation, visually, with imaging techniques, endoscopically assisted or using open surgical techniques. Manipulative factors 12 can be introduced or applied, typically at one or both ends of the conduits 142, 144 with external (percutaneous) connections of the tubes, conduits or threads. The outer barrier or sheath of the tube (equivalent to the plate described above) and the makeup of the inner core (equivalent to the scaffolding described above) depend on the therapy intentions and the method of introduction, including placement, manipulation and control. With the system in a tubular configuration, the outer barrier is also the contact layer.

The tube can be placed in solid tissue, such as muscle or the liver using imaging techniques with a series of guide wires, followers and dilators, similarly to techniques for endovascular access. In long muscles such as the quadriceps, both entrance and exit areas are more feasible and more easily accomplished with a single guide wire or thin trocar. Input and output can thus be provided at opposite poles as the simplest and most efficient system for fluid manipulation. For example, in the liver, without open or endoscopic assistance, a single external conduit could serve as both input and output ports by alternating the functions or by use as a conduit carrying side-by-side smaller input/output lines that would travel in a preconfigured fashion through the outer sheath and inner core whereby the input would be instilled at one end and the output would be withdrawn from the opposite end and these functions could travel side-by-side in the single conduit separately contained.

Once the tube, conduit or thread has been placed, a series of rinses alternating with suction would be instituted to clear the space of the debris of the trauma of placement and to draw the surrounding tissue tightly against the thread and then to stimulate neovascular ingrowth to start. The outer sheath could have a pore size sufficient to be able to remove the blood and cell damage from placement. This could take an estimated one to two days or until the effluent is clear. The cell seeding then starts and is continued until it also comes out the effluent. The inner core is a scaffolding material that is biodegradable and chosen for its affinity to the cells to be seated. The outer sheath is in removed and the inner core, now seeded with cells, is left in place to grow and “take” as a graft of bioengineered tissue grown in-situ. If a single port is used, the inner core can be cut below the skin line and allowed to retract. If a double (2-ended) port is used, the output port is cut below the skin line and the outer sheath is then pulled out through the outer port. If the core is in the port, it is also cut off below the skin and allowed to retract. The end result is that the nonabsorbable outer sheath is removed and the absorbable scaffolding is left in a subcutaneous (inter-tissue) position.

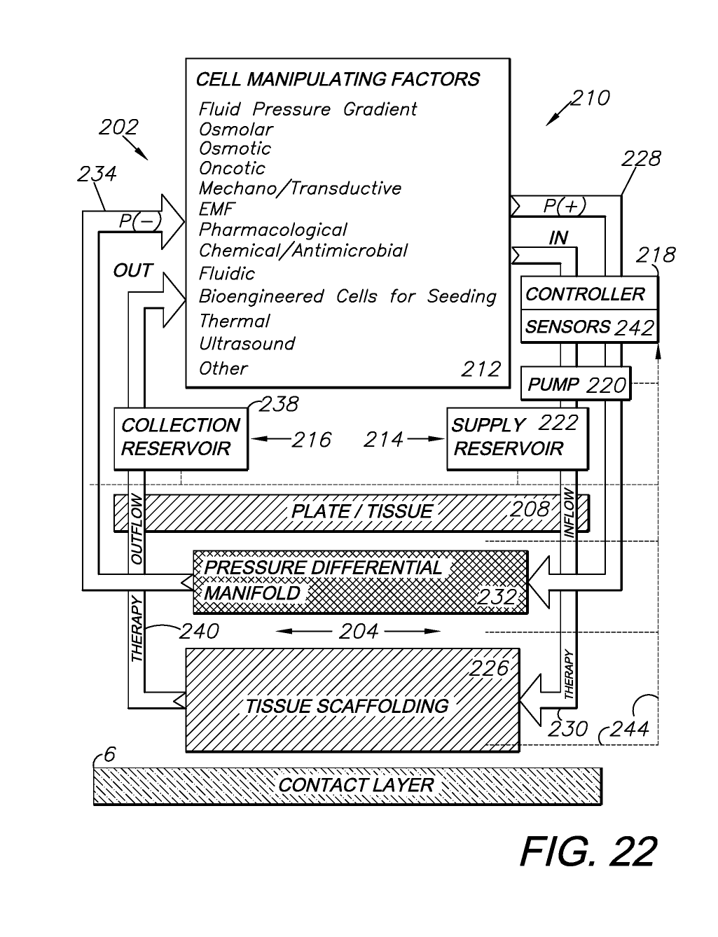

FIG. 22 shows a modified control system 202 comprising an alternative aspect of the present invention. The system 202 includes an inter-tissue space/therapy zone 204, which also defines a flow layer(s) for fluids generated internally and/or introduced externally. The tissue contact layer 6 can be located anywhere appropriate for treatment with the systems 2 and 202, including subdermal, subcutaneous, externally and internally; and in or on body cavities, organs, muscle fibers, ligamentous and osseous (skeletal) structure, etc. A plate/tissue component 208 can comprise a physical structure, such as a biocompatible material adapted for placement in or on the therapy zone 204. Alternatively, the component 208 can comprise a patient's tissue layer, such as the dermis, epidermis, etc. Functionally the component 208 cooperates with a pressure differential manifold 232 to facilitate and direct the flow of fluid, microbial agents, medications, irrigation, and other substances in the therapy zone 204. Either or both of the tissue scaffolding 226 and the pressure differential manifold 232 can comprise cellular matrices, synthetic tissue, living tissue or derivatives of living tissue.

The system 202 can include a variety of configurations with the plate/tissue component 208 cooperating with the manifold 232 and scaffolding 226 to form the pressure differential zone 204. Fluid pulse waves can be introduced to the therapy zone 204 by cycling a pump 220 with a controller 218 and pulsing fluid through various tubing and manifold configurations, including those shown in FIGS. 2-21. A sensor suite 242 is connected to the controller 218 and can include multiple sensor suite feeds 244 extending to various components and areas of the therapy zone 204. The sensor suite 242 can include sensors for monitoring various operating parameters, including pressure, temperature, microbial activity, chemical composition (e.g., oxygen and CO₂ levels), etc. Sensor inputs to the controller 218 can be digitized for processing by the microprocessor controller 218. The sensor signal input information can be utilized by the controller 218 for controlling various operating parameters of the system 202, such as the pump 220, the inflow/outflow lines 230/240 and the factor source 212.

The tubing and manifold elements shown therein can be rearranged and reconfigured as necessary to achieve a wide range of alternative systems for accommodating various patient conditions and treatment objectives.

Relatively small-amplitude pressure changes of, for example, a few mm Hg, can be sufficient for achieving desired therapeutic results. More specifically, such pressure changes can stimulate cellular activity, reepithelialization, cell migration, regeneration and other physiological changes associated with the healing process. Alternatively or additionally, components of the system 202, such as the bellows-equipped pillars 122 shown in FIG. 19, can provide or supplement such pressure waves, for example with the blood pressure cycles of the circulatory system or similar pressure-varying, dynamic physiological functions, such as musculature, lymphatic, respiratory, etc. The system 202 can thus operate using the dynamic pulsations naturally occurring in-vivo, and/or with externally-applied forces, such as the pump 220.

In addition to the in-vivo systems and methodologies described herein, the system 202 is adaptable for benchtop, tissue culture, tissue engineering, ex-vivo and other applications for a wide range of research, bioengineering, tissue culture and other useful applications, which share a common element of cellular control and manipulation.

A general interface 210 can comprise a wide range of suitable component/patient interface constructions, such as internal/external dressings, closure screens, etc. For examples, see Zamierowski U.S. Pat. No. 4,969,880; No. 5,100,396; No. 5,261,893; No. 5,527,293; and No. 6,071,267; and U.S. Patent Publications No. 2008/0228221 and No. 2008/0228222, which are incorporated herein by reference. An exemplary list of cell manipulating factors as shown at 212 for application to the therapy zone 204 via the interface 210, and is not to be construed as limiting. Various other cell manipulating factors can be employed for achieving desired therapeutic and other beneficial results. On a supply/input side 214 of the system 202, a controller 218 can be provided for preprogramming to control various components and operating parameters of the system 202, such as a pump 220 for delivering fluids and other factors from the source 212 to the pressure differential manifold 232 via inlet lines 228 and to tissue scaffolding 226 via therapy inflow input lines 230. Likewise on the outlet side 216, line 234 is connected to the pressure differential manifold 232 and returns to the source 212. The therapy outflow line 240 is connected to the tissue scaffolding 226 and returns to the source 212.

An optional supply reservoir 222 can be connected to the therapy inflow line 230 and can provide a secondary or alternative source of pharmacological and other factors for input to the therapy zone 204 via the therapy inflow line 230. A corresponding collection reservoir 238 can receive fluid from the therapy zone 204 via the therapy outflow line 240. Of course, collected waste fluid can be disposed of using established medical waste disposal procedures.

These systems 2 and 202 shown and described above comprise exemplary aspects of the invention, which may be embodied in various other forms. For example, the planar orientations of the system components can be rearranged and reconfigured in-situ as determined by the medical practitioner. Alternative orientations can include inverted, vertical, horizontal, etc. Moreover, the orientations discussed above are for illustration and could vary depending upon the position of the patient. Still further, the pressure differential manifold 232 could be formed within or below the tissue scaffolding 226 and in various spatial relationships to the plate/tissue 208. The component configurations can assume any appropriate configuration, such as tubular, spiral, circular, etc.

### II. Bio-Reactor Embodiments

A bio-reactor (or bioreactor) is defined as any system or device which supports a biologically active environment. In this context, bio-reactor refers to a device or system for promoting the growth of cells and tissues. An example of a bio-reactor, which shall not be limiting, is a tissue scaffolding, as described above. Cell growth promoters may be introduced into the body and can include any treatments which aid in cell or tissue growth. Such cell growth promoters may include, but are not limited to, cells introduced for cell seeding, biomaterials for tissue engineering, pharmacological drugs, pressure waves, vitamins, nutrients, etc. In an embodiment of the present invention, a bio-reactor is configured for introducing and/or releasing cell growth promoters into a therapy zone. In a preferred embodiment of the present invention, bio-reactors are used to aid in tissue engineering. This may include uses clinically, for diagnosis, for lab testing, for assessment, or for other medical testing or procedures. Alternatively, the bio-reactor may be used to aid in wound healing. Most commonly, the bio-reactors of the present invention are used in vivo in combination with a force transducer. However, these bio-reactors may alternatively be used in vivo without a force transducer, in situ, or in vitro.

In an embodiment of the present invention, an implanted bio-reactor is configured for placement in combination with a force transducer to promote cellular activity for tissue engineering. The combination of bio-reactor and force transducer produces a therapy zone with enhanced fluid dynamics surrounding the bio-reactor. The force transducer can be any device configured for providing cell-manipulating factors or forces to the therapy zone. The cell-manipulating factors or forces may include, but are not limited to, physical contact; fluidic contact; fluid pressure gradient; pressure wave; osmolar pressure; osmotic pressure; oncotic pressure; mechano/transductive; electromagnetic force (EMF); pharmacological; chemical; antimicrobial; fluidic; bioengineered for seeding; thermal; acoustic; and/or ultrasound (sonar).

The bio-reactor of the present invention can be configured for implantation within many different tissues in the body. For example, in one embodiment, a bio-reactor can be percutaneously implanted within the subcutaneous layer. Alternatively, a bio-reactor can be placed remotely within a solid organ. In another embodiment, a bio-reactor can be endoscopically placed within a hollow viscous. Optionally, one or more additional, or secondary, bio-reactors can be implanted in proximity to the initial bio-reactor, creating respective secondary therapy zones with enhanced fluid dynamics.

Many different configurations of force transducers embody the present invention. One exemplary embodiment of a force transducer of the present invention is a negative pressure, or suction, surface device configured for placement over an intact tissue surface. The intact tissue surface may be intact skin or an internal tissue surface. In this embodiment, a bio-reactor is implanted beneath the negative pressure surface device. The negative pressure surface device, in this embodiment, is configured to provide negative pressure waves, or suction, to a therapy zone surrounding the bio-reactor.

One common embodiment includes a negative pressure surface device placed over intact skin with a bio-reactor percutaneously implanted within the underlying subcutaneous layer. However, the present invention can be configured for many other placements and applications of surface devices and bio-reactors. An embodiment would be internal visceral placement of a bio-reactor and visceral surface placement of a compressive surface device. Examples include internal liver implantation of the bio-reactor and surface placement of the compressive device, vacuum being achieved, and internal solutions in addition to the implant being achieved by tubing led through the skin surface and controlled exterior to the body. Alternatively, the entire system could be designed to be implanted and self-contained. The implantation and removal process could all be done laparoscopically with minimal incisions. This same process could be applied to intramural intestinal implantation, or any other hollow viscera, and a serosal or external visceral compression, or negative pressure, device. This approach may also be applied to any solid viscera such as the spleen or kidney. It can be applied to muscle and muscular organs such as the heart, fascial linings, and any intramuscular implantation.

In addition to the aforementioned embodiments, some specific embodiments with a negative pressure surface device and an implanted bio-reactor include a visceral serosa negative pressure surface device and a subserosal, intra-visceral, or intra-luminal bio-reactor. The present invention can be applied with an extra-osseous surface device and an intra-osseous bio-reactor. Another embodiment includes a fascial or skin negative pressure device and a sub-fascial bio-reactor. A negative pressure surface device can be applied to a cardiac surface with an intra-cardiac bio-reactor. Another embodiment includes a muscular surface device and an intra-muscular bio-reactor. The surface device can be applied to the belly wall with an intra-peritoneal bio-reactor. Alternatively, the negative pressure surface device can be applied to the chest wall with an intra-thoracic bio-reactor. The surface device could also be applied to a neuronal surface, nerve wall, or spine wall with an intra-neural bio-reactor. These embodiments are exemplary applications of the present invention, but they are not limiting. The surface device can be applied to any bodily surface with a corresponding bio-reactor implanted in tissue.

Alternatively, the force transducer may consist of a device other than a negative pressure surface device. A force transducer of the present invention may include any device configured for supplying one or more cell growth promoters or cell-manipulating factors to a therapy zone surrounding a bio-reactor. In one configuration, a force transducer is remote from a bio-reactor and therapy zone and configured for being controlled outside of the body. In such an embodiment, implantable tubing is led from the force transducer into the body and to the therapy zone. This tubing is configured to introduce and supply cell-manipulating factors and/or forces to the therapy zone surrounding the bio-reactor.

In another embodiment, a force transducer is locally present in proximity to a bio-reactor and surrounding therapy zone. In this embodiment, the force transducer is configured for implantation in proximity to the therapy zone. The force transducer controls the supply of cell-manipulating factors to the therapy zone, internally. Optionally, multiple force transducers may be utilized, each supplying cell-manipulating factors and/or forces to the therapy zone. If multiple force transducers are included in the system, one or more may be implanted with internal controls, and one or more may be remote, with tubing connected, and controlled from outside the body. Another embodiment includes one force transducer comprising a negative pressure surface device and a second force transducer configured to supply one or more additional cell-manipulating factors and/or forces to the therapy zone.

The present invention can be positioned so that a natural tissue layer acts as a reflector, or plate, which reflects and amplifies pressure waves within the therapy zone. Some examples of natural tissue layers which could act as a natural reflector include, but are not limited to, bone tissue, organ walls, muscle tissue, etc. However, in configurations where a natural reflector is not possible or practical, a concave-curved, internal reflector may be implanted as part of the tissue engineering system. The internal reflector is configured for implantation in proximity to the bio-reactor and for amplifying or optimizing pressure waves within the therapy zone surrounding the bio-reactor. In addition to multiple force transducers, multiple bio-reactors and/or multiple implantable internal reflectors can be included in embodiments of the present invention in order to optimize fluid dynamics in the therapy zone and respective secondary therapy zones.

### III. Negative Pressure Surface Device and Implanted Bio-Reactor

In an embodiment of the present invention, a surface device or dressing 303 of the negative pressure variety is used in conjunction with a bio-reactor 320 to be implanted within tissue. In one preferred embodiment, the negative pressure surface device is configured to be sized longer and wider than, or beyond the boundaries of, the implanted bio-reactor. For example, if the length and width of the bio-reactor 320 is a 1 in.×1 in. square, the surface dressing 303 may have a length and width forming a 4 in.×4 in. square. However, it is to be recognized that there can be a wide variation in these proportions.

First the bio-reactor 320 is implanted, ideally from a distant approach site rather than from beneath the surface portion of the dressing 303. Rolled implantable devices of permanent and biodegradable materials have been previously described as bio-reactors 320 so that implantation and addition of cells and solutions can be carried out by needle or catheter, left permanently or removed after instillation. An incision and surgical placement can also be utilized. The negative pressure surface device of this embodiment is configured for placement over intact skin, providing negative pressure to a therapy zone to aid in tissue engineering. However, alternatively, the surface dressing can be placed over a wound or incision, with a bio-reactor implanted below the wound or incision, to aid in wound healing.

The dressing 303 may be made up of one or more layers of finely woven (minimal interstices size) fabric, gels, colloid, or closely compressed smooth foam with minimal, if any, pore size. The dressing 303 is placed on the skin and covered by a material that serves as a manifold and compressible core 308 which produces closely held compression of the contact layer to the skin. Reticulated open-cell foam is the most commonly used material, but any other porous, compressible material can be used. This compressible core is covered by a drape or cover layer 304 that either has minimal or a low vapor transmission rate so that a small vacuum source can easily exceed it and produce a continuous collapse of this dressing and the desired compression effect.

This combination of implanted bio-reactor 320 and overlying surface compression device 303 produces a therapy zone 326 with enhanced fluid dynamics which enhances the rate of activity in or on the bio-reactor 320. In a preferred embodiment, the surface device or dressing is configured for placement over intact tissue. Alternatively, the negative pressure dressing can be used over a wound or incision. In an embodiment with a surface device 303 attached to the epidermis 334 and a bio-reactor 320 embedded within the subcutaneous layer 338, a primary therapy zone 326 is formed including portions of the epidermis 334, dermis 336, and subcutaneous layer 338 surrounding the bio-reactor 320 and surface device 303. Optionally, one or more additional bio-reactors can be used in conjunction with the primary bio-reactor 320 and surface device 303, forming additional therapy zones.

FIG. 23 shows an embodiment of a bio-reactor engineering or wound (BREW) treatment system 302 of the present invention including a negative pressure surface device 303 and an implanted bio-reactor 320. In this embodiment, the surface dressing 303 is configured for placement over intact skin. The dressing 303 includes a compressible core 308; an overdrape or cover layer 304 configured for releasable attachment to a patient's skin or epidermis 334; a wicking liner; and an output port 310 configured for attachment to a negative pressure source. The compressible core 308 can be made of open-cell, reticulated foam or another material capable of compression under negative pressure. The wicking liner may be made up of rayon or any other suitable material with absorbent and/or wicking capabilities. A bio-reactor 320, in this embodiment, is configured for implantation beneath the surface device 303 within the subcutaneous layer 338. In this embodiment, the bio-reactor 320 is configured to be implanted through an implantation route 318 which begins remotely from a therapy zone 326 where the bio-reactor 320 is to be placed. Negative pressure applied through the surface device 303 in combination with the implanted bio-reactor 320 creates a primary therapy zone 326 with enhanced fluid dynamics, promoting increased activity in the bio-reactor 320. FIG. 23 also shows an optional secondary bio-reactor 370, which can be embedded in the subcutaneous or another layer via a secondary implantation route 368, creating a secondary therapy zone 376 with enhanced fluid dynamics. Any number of additional bio-reactors and therapy zones can be utilized, as desired, to enhance cell and tissue growth.

### IV. Dynamic Bio-Reactor Engineering or Wound (BREW) Treatment Embodiments

A bio-reactor engineering or wound (BREW) treatment system can be made dynamic with the integration of an ex-vivo component configured for providing external pressure waves, in addition to the patient's internal pulsations, via an impulse delivery system. The ex-vivo component can be a force transducer 424 capable of producing control factors or forces 440 including, but not limited to, physical contact; fluidic contact; fluid pressure gradient; pressure wave; osmolar pressure; osmotic pressure; oncotic pressure; mechano/transductive; electromagnetic force (EMF); pharmacological; chemical; antimicrobial; fluidic; bioengineered for seeding; thermal; acoustic; and/or ultrasound (sonar). The control factors/forces 440 interact with an implanted bio-reactor 420 and tissue within a therapy zone 426 surrounding the bio-reactor 420. The force transducer may be controlled outside the body and attached to implantable tubing 422 configured for delivering cell manipulating factors and/or forces to the therapy zone. Alternatively, the force transducer can be implanted near the bio-reactor, with internal release of cell manipulating factors and/or forces.

This embodiment allows for differential treatment of the therapy zone 426, with the additional dynamic forces or factors 440 affecting the activity of the bio-reactor 420. The external forces 440, in addition to the body's internal pulse waves, induce increased activity of the bio-reactor 420. These additional waves 440 are capable of providing enhanced fluid dynamics in a therapy zone 426, which, in this embodiment, includes portions of the epidermis 434, dermis 436, and subcutaneous layer 438 surrounding the bio-reactor 420. Enhanced fluid dynamics produce enhanced activity in the bio-reactor 420. Such a dynamic ex-vivo component 424 capable of producing different types of pressure waves allows for testing different forces to establish optimum fluid dynamics, which can depend on the make-up and location of the bio-reactor 420 and the individual patient's responsiveness to different pressure waves.

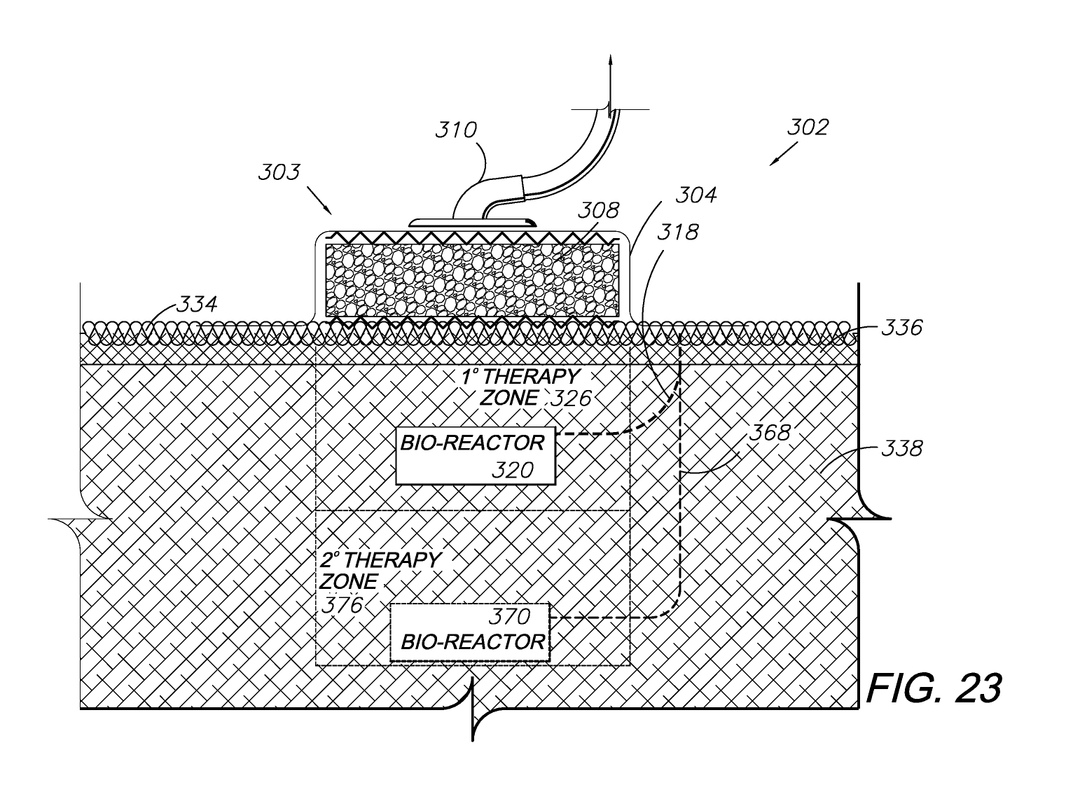

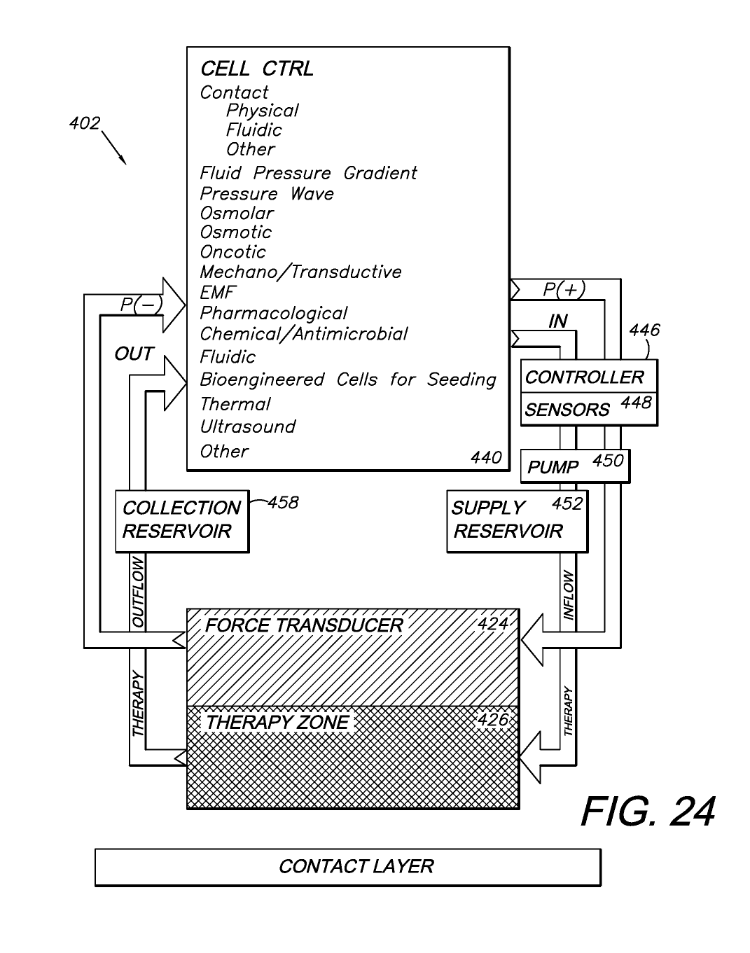

FIG. 24 shows a schematic block diagram of an embodiment of a dynamic bio-reactor engineering or wound (BREW) treatment system 402 including a force transducer 424 configured to apply one or more control factors and/or forces 440 to a therapy zone 426, the therapy zone 426 including at least one bio-reactor 420. The dynamic BREW system 402 includes a controller 446, sensors 448, a pump 450, a supply reservoir 452, and a collection reservoir 458 for sensing and controlling the inflow and outflow of control factors/forces 440 to and from the therapy zone 426.

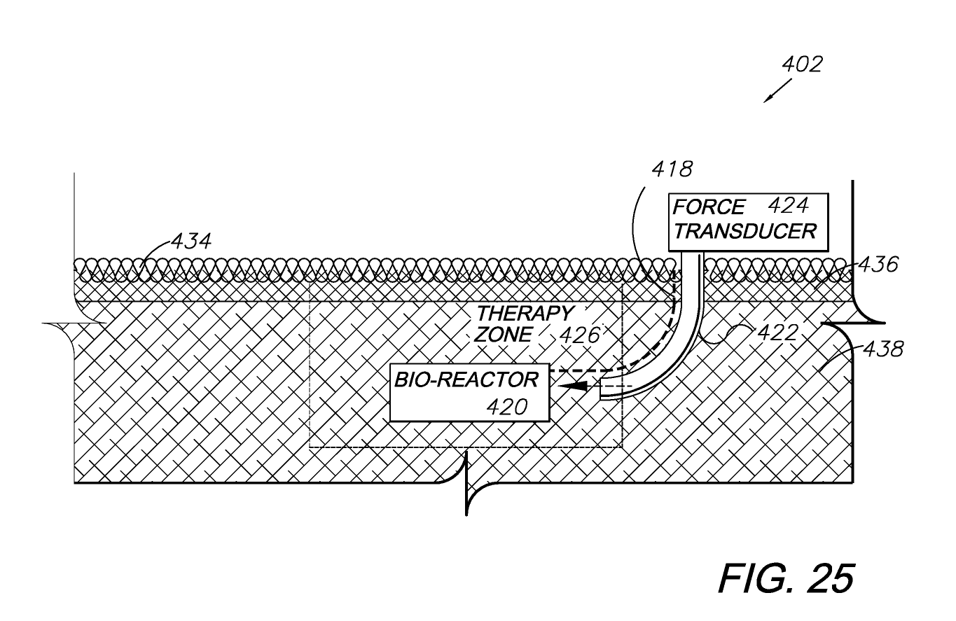

FIG. 25 shows the dynamic bio-reactor engineering or wound (BREW) treatment system 402 including a force transducer 424 configured for supplying cell manipulating factors and/or forces to a therapy zone 426 surrounding an implanted bio-reactor 420. In this embodiment, the force transducer 424 is configured for being controlled remotely from the therapy zone 426, outside the body. Implantable tubing 422 is connected to the force transducer 424 and configured for implantation into the body and for supplying cell manipulating factors and/or forces to the therapy zone 426. The bio-reactor 420 and tubing 422 are configured to be implanted through the epidermis 434, the dermis 436, and into the subcutaneous layer 438 via an implantation route 418.

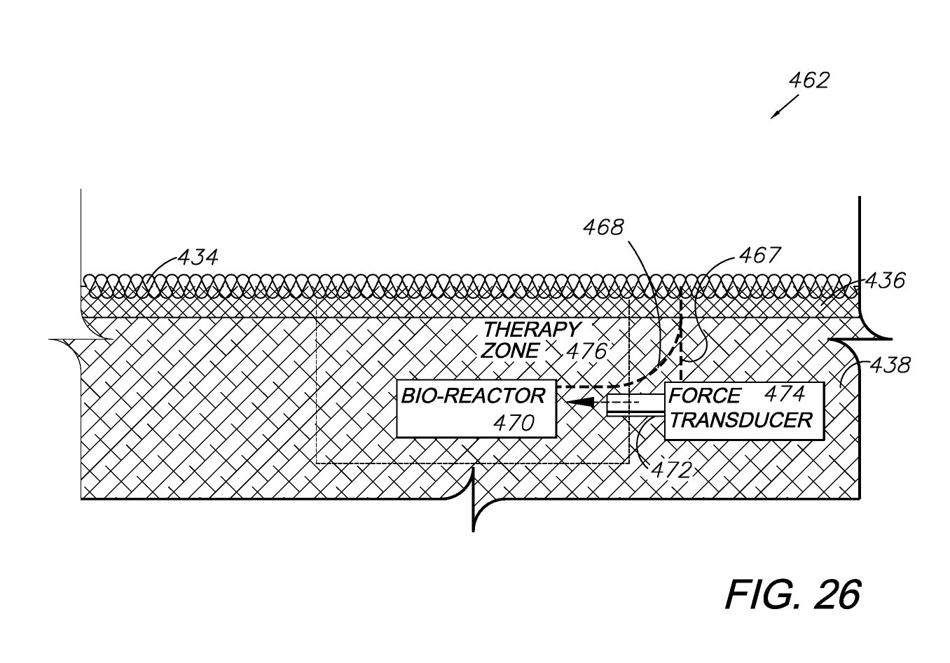

FIG. 26 shows an alternative embodiment of a dynamic bio-reactor engineering or wound (BREW) treatment system 462 including a force transducer 474 and a bio-reactor 470. In this embodiment, the force transducer 474 is configured to be implanted in the subcutaneous layer 438 and for locally controlling the release of cell manipulating factors and/or forces to a therapy zone 476 surrounding the bio-reactor 470. The bio-reactor 470 is configured for being implanted remotely through the epidermis 434, the dermis 436, and into the subcutaneous layer 438 via a bio-reactor implantation route 468. The force transducer 474 is configured for being implanted through the epidermis 434, the dermis 436, and into the subcutaneous layer 438 via a force transducer implantation route 467. Tubing 472 is connected to the implanted force transducer 474 and configured for supplying cell manipulating factors and/or forces to the therapy zone 476.

In an embodiment of the present invention, a negative pressure surface dressing or device can be used in combination with an implanted bio-reactor and a second force transducer. In this embodiment, the bio-reactor is configured for implantation within tissue, and the surface dressing is configured for placement over intact tissue and for providing negative pressure to a therapy zone surrounding the bio-reactor. The second force transducer is configured for supplying one or more additional cell manipulating factors and/or forces to the therapy zone.

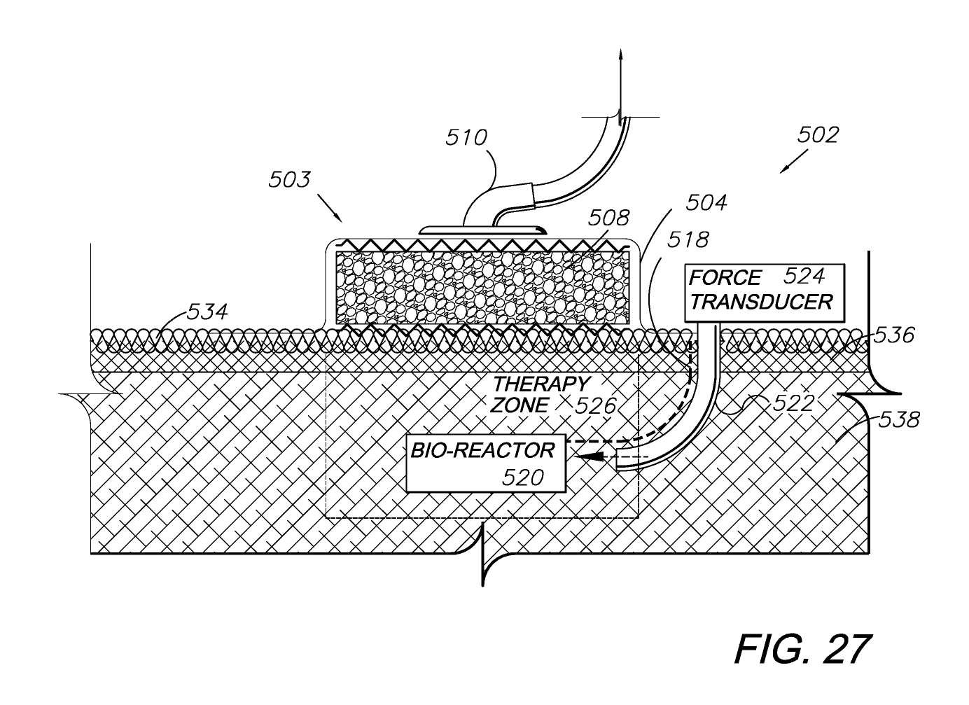

FIG. 27 shows an embodiment of a dynamic bio-reactor engineering or wound (BREW) treatment system 502 including a negative pressure surface device 503, an implanted bio-reactor 520, and a second force transducer 524 configured to provide external control factors and/or forces. In the embodiment shown in FIG. 27, the surface dressing 503 is configured for placement over intact skin. Alternatively, a surface dressing can be placed over a wound or incision with a corresponding bio-reactor implanted below and a second force transducer. The dressing 503 includes a compressible core 508; an overdrape or cover layer 504 configured for releasable attachment to a patient's skin or epidermis 534; a wicking liner; and an output port 510 configured for attachment to a negative pressure source. The bio-reactor 520, in this embodiment, is configured for implantation beneath the surface device 503, in the subcutaneous layer 538. The second force transducer 524 provides external pressure waves to a therapy zone 526 including portions of the epidermis 534, dermis 536, and subcutaneous layer 538 surrounding the bio-reactor 520. In this embodiment, the second force transducer 524 is configured for being controlled remotely from the therapy zone 526, outside the body. Implantable tubing 522 is connected to the second force transducer 424 and configured for supplying the external factors and/or forces 440 to the therapy zone 426. The bio-reactor 520 and tubing 522 are configured for implantation along an implantation route 518 which begins remotely from the therapy zone 526. The combination of negative pressure, a bio-reactor 520, and another force transducer 524 providing control factors/forces provides further enhanced fluid dynamics, promoting increased activity in the bio-reactor 520.

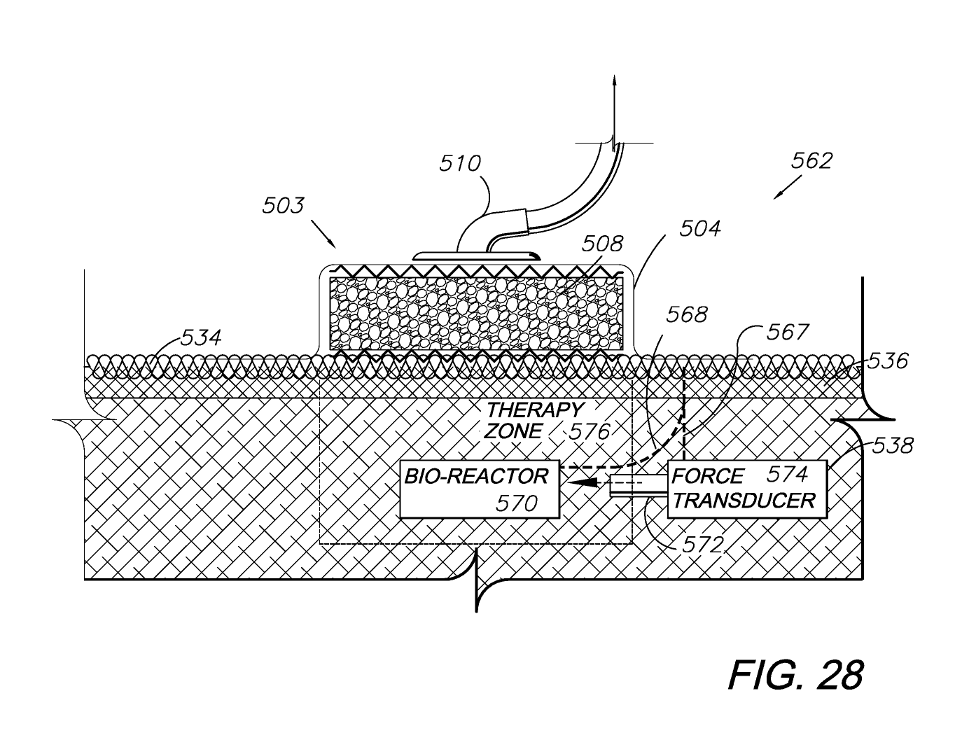

FIG. 28 shows an alternative embodiment of a dynamic bio-reactor engineering or wound (BREW) treatment system 562 including a negative pressure surface device 503, a bio-reactor 570, and a second force transducer 574. In this embodiment, the force transducer 574 is configured to be implanted in the subcutaneous layer 538 and for locally controlling the release of cell manipulating factors and/or forces to a therapy zone 576 surrounding the bio-reactor 570. The bio-reactor 570 is configured for being implanted remotely through the epidermis 534, the dermis 536, and into the subcutaneous layer 538 via a bio-reactor implantation route 568. The force transducer 574 is configured for being implanted through the epidermis 534, the dermis 536, and into the subcutaneous layer 538 via a force transducer implantation route 567. Tubing 572 is connected to the implanted force transducer 574 and configured for supplying cell manipulating factors and/or forces to the therapy zone 576.

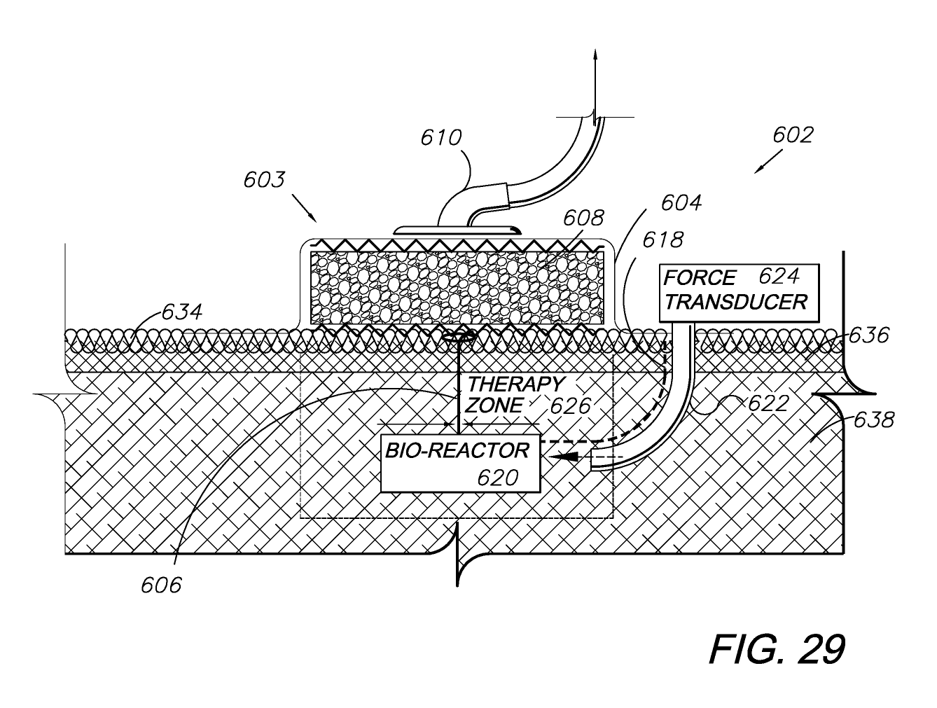

FIG. 29 shows an embodiment of a dynamic BREW therapy system 602 configured to aid in wound healing. The dynamic BREW therapy system includes an implanted bio-reactor 620, a negative pressure surface device 603, and a second force transducer 624. In this embodiment, the negative pressure dressing 603 is configured for placement over a wound or incision 606. The dressing 603 includes a compressible core 608; an overdrape or cover layer 604 configured for releasable attachment to a patient's skin or epidermis 634; a wicking liner; and an output port 610 configured for attachment to a negative pressure source. The bio-reactor 620, in this embodiment, is configured for implantation beneath the wound or incision 606 and the dressing 603. The second force transducer 624 is configured for providing cell manipulating factors and/or forces to a therapy zone 626 including portions of the epidermis 634, dermis 636, and subcutaneous layer 638 surrounding the wound 606 and bio-reactor 620. In this embodiment, the second force transducer 624 is configured for being controlled remotely from the therapy zone 626, outside the body. However, in alternative embodiments, a second force transducer can be implanted in proximity to the therapy zone and configured for controlling local release of factors and/or forces to the therapy zone. Implantable tubing 622 is connected to the second force transducer 624 and configured for supplying the external factors and/or forces to the therapy zone 626. The bio-reactor 620 and tubing 622 are configured for implantation along an implantation route 618 which begins remotely from the therapy zone 626. This combination of negative pressure, a bio-reactor 620, and another force transducer 624 providing control factors/forces provides enhanced fluid dynamics, promoting increased activity in the bio-reactor 620 and aids wound healing.

### V. Dynamic Bio-Reactor Engineering or Wound (BREW) Treatment with Internal Concave Reflector Embodiment

Another embodiment of a BREW system 702 includes an internal, concave-curved, or parabolic-shaped, reflector 728 in addition to an implanted bio-reactor 720, a negative pressure surface device 703, and a dynamic ex-vivo component or force transducer 724 configured for providing external control factors or forces to a therapy zone 726. The internal reflector 728 is configured to be positioned below the bio-reactor 720, and such positioning effectively amplifies or maximizes primary waves within the therapy zone 726. Pressure waves hitting a concave surface cause the waves to be reflected off of the surface and amplified. Placing a concave internal reflector 728 underneath an implanted bio-reactor 720 amplifies the waves produced by the ex-vivo component 724 as well as internal bodily pulse waves, ultimately maximizing or optimizing the fluid dynamics surrounding the bio-reactor 720. Enhanced fluid dynamics as a result of the ex-vivo dynamic component 724 and the internal reflector 728 increase the cellular activity on or within the bio-reactor 720.

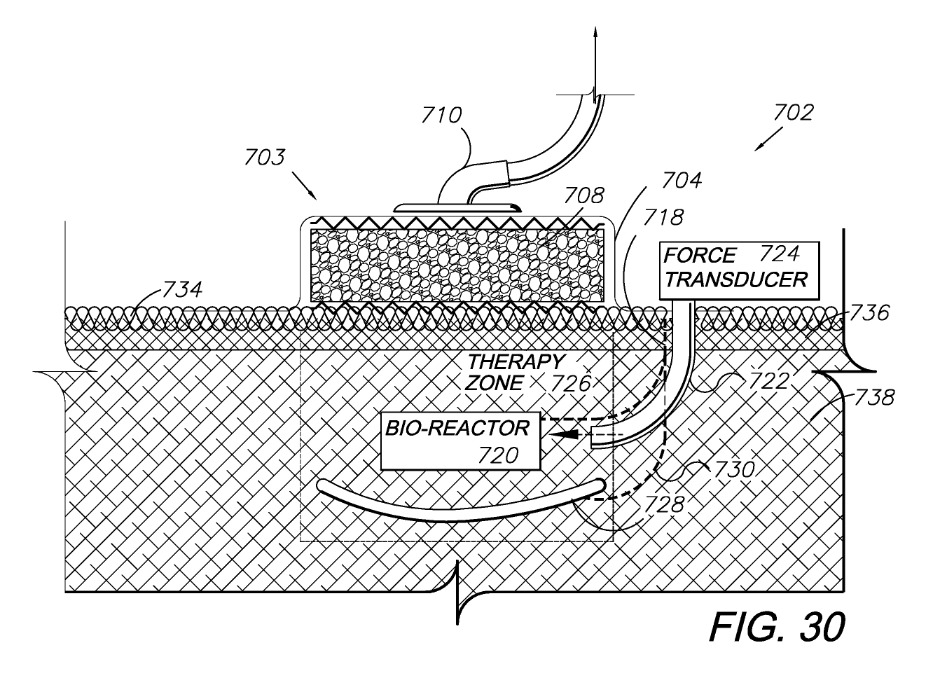

FIG. 30 shows an embodiment of a dynamic BREW system 702 including a negative pressure surface device 703, an implanted bio-reactor 720, a second force transducer 724 configured to provide external control factors and/or forces, and a concave internal reflector 728 configured to reflect force waves. The surface dressing 703, in this embodiment, is configured for placement over intact skin. Alternatively, the dynamic BREW system 702 could be used to aid in wound healing. The dressing 503 includes a compressible core 708; an overdrape or cover layer 704 configured for releasable attachment to a patient's skin or epidermis 734; a wicking liner; and an output port 710 configured for attachment to a negative pressure source. The bio-reactor 720, in this embodiment, is configured to be implanted beneath the negative pressure surface device 703, in the subcutaneous layer 738. The second force transducer 724 provides external pressure waves to a therapy zone 726 including portions of the epidermis 734, dermis 736, and subcutaneous layer 738 surrounding the bio-reactor 720. In this embodiment, the second force transducer 724 is configured for being controlled remotely from the therapy zone 726, outside the body. However, in alternative embodiments, a second force transducer can be implanted in proximity to the therapy zone and configured for controlling the local release of factors and/or forces to the therapy zone. Implantable tubing 722 is connected to the second force transducer 724 and configured for supplying external factors and/or forces to the therapy zone 726. The bio-reactor 720 and tubing 722 are configured for implantation along an implantation route 718 which begins remotely from the therapy zone 726. The internal reflector 728 is configured to be implanted beneath the bio-reactor 720 in the subcutaneous layer 738 via an internal reflector implantation route 730. The reflector 728 reflects pressure waves in the therapy zone 726, allowing pressure waves to be maximized or optimized for activity in the bio-reactor 720. This combination of negative pressure, a bio-reactor 720, a second force transducer 724 providing external control factors/forces, and a concave internal reflector 728 provides even further enhanced fluid dynamics, promoting increased cell and tissue growth via the bio-reactor 720.

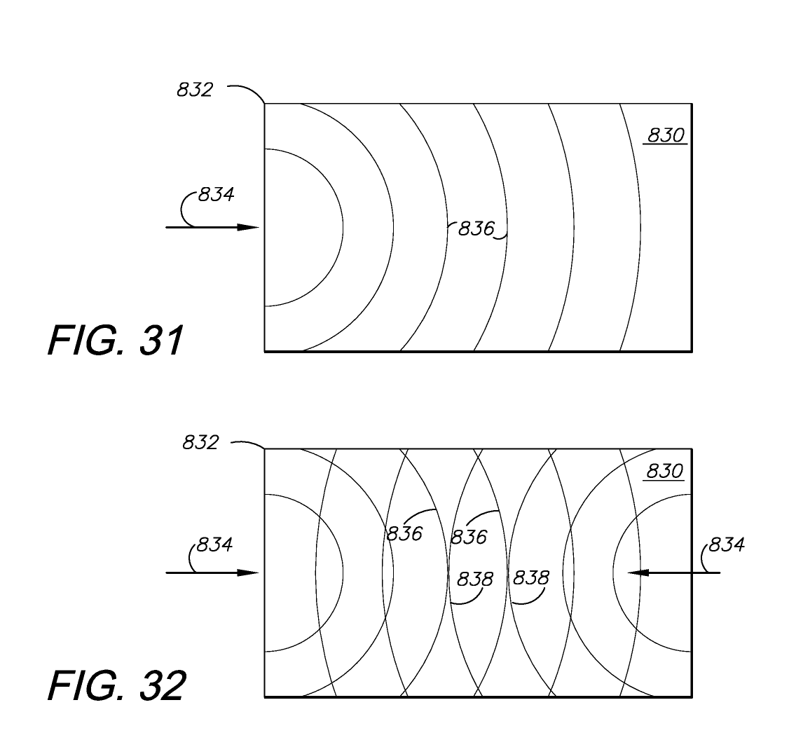

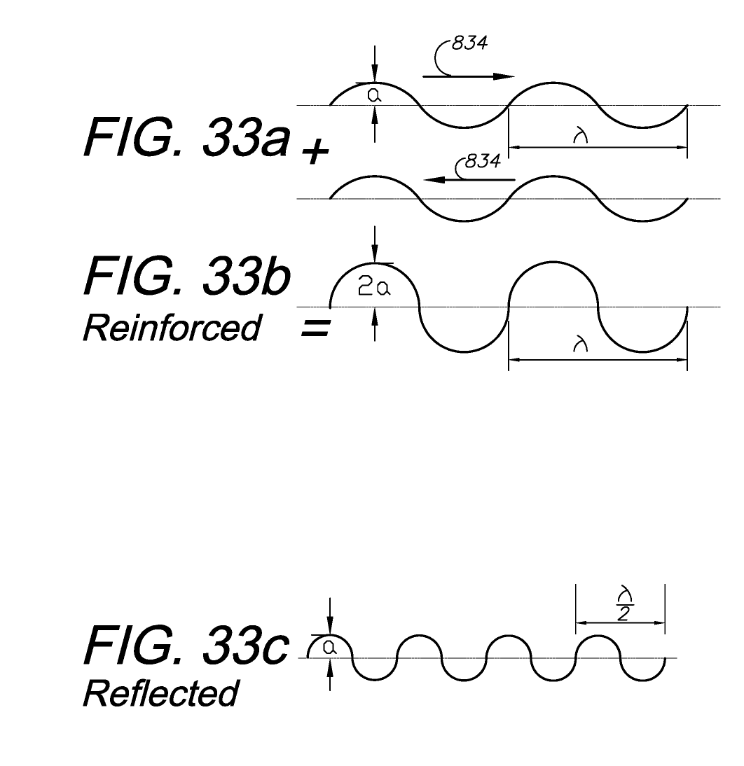

FIGS. 31-33c show general principles of pressure wave reflection. In embodiments of the present invention, pressure wave reflection may occur based on positioning of the present invention in relation to natural tissue layers. Alternatively, an implanted internal reflector 728 may cause pressure wave reflection. FIG. 31 shows a pressure wave medium 830 within a container 832, which can be subject to an external force depicted by a force arrow 834. Pressure waves 836 are shown within the container 832. FIG. 32 shows the container 832 with reflected pressure waves 838 in the medium 830. FIG. 33a shows combining synchronized pressure waves, which can be moving in opposite directions indicated by arrows 834, resulting in a reinforced pressure wave, as shown in FIG. 33b, with twice the amplitude but the same wavelength and frequency. FIG. 33c shows a reflected wave consideration with twice the frequency, half the wavelength, and the same amplitude.

Incident and reflected waves can be combined at various angles and timing with the use of an internal reflector 728 for achieving desired pressure wave effects. For example, enhanced pressure waves can aid in enhancing tissue growth, tissue regeneration, tissue healing, blood circulation, and lymphatic fluid circulation.

It is to be understood that while certain aspects and embodiments of the invention are described and shown, the invention is not limited thereto and can assume a wide range of other, alternative aspects and embodiments.

Having thus described the disclosed subject matter, what is claimed as new and desired to be secured by Letters Patent is:

1. A medical cellular factor control system for a tissue therapy zone, which system comprises:
a source of a cell-manipulating factor;
a factor placement device connected to said factor source and configured for placing said factor in tissue in said therapy zone, said factor placement device including a force transducer configured for applying a force in said therapy zone;
a non-dermal derived tissue scaffolding configured for implantation within in-vivo tissue in said therapy zone; and
wherein said tissue scaffolding is configured for enhancing cellular-level reactions within said therapy zone.

2. The medical cellular factor control system according to claim 1, which includes:
said force transducer including a differential fluid pressure device configured for providing pressure impulses within said therapy zone for factor placement and cellular manipulation.

3. The medical cellular factor control system according claim 2, which includes:
an ex vivo fluid pressure control device; and
said fluid pressure control device configured for pulsing fluid pressure in vivo.

4. The medical cellular factor control system according to claim 3, which includes:
said fluid pressure control device including an external pump; and
a fluid conduit configured for ex vivo connection to said pump and in vivo connection to said non-dermal derived tissue scaffolding and communicating fluid between said pump and said scaffolding.

5. The medical cellular factor control system according to claim 1, wherein said factor is chosen from among the group comprising: physical contact, fluidic contact, fluid pressure gradient, pressure wave, osmolar, osmotic, oncotic, mechano/transductive, electromagnetic force (EMF), pharmacological, chemical, antimicrobial, fluidic, bioengineered cells for seeding, thermal, acoustic, and ultrasound.

6. The medical cellular factor control system according to claim 2, wherein said force transducer includes a pressure differential manifold configured for placement in vivo in said therapy zone.

7. The medical cellular factor control system according to claim 2, which includes:
said impulses comprising systolic/diastolic in vivo blood pressure differentials; and
said force transducer configured for stimulating cellular activity in said therapy zone with in vivo blood pressure pulsations.

8. The medical cellular factor control system according to claim 1 wherein:
said scaffolding structure includes a cell growth promoter chosen from among the group consisting of: cells for cell seeding, biomaterials for tissue engineering, pharmacological drugs, vitamins, and nutrients;
said scaffolding structure is biodegradable or biocompatible; and
said structure is configured for remote implantation.

9. The medical cellular factor control system according to claim 1, wherein said force transducer includes a concave-curved internal reflector configured for placement in said therapy zone and amplifying pressure waves therein.

10. A medical cellular factor control system for a tissue therapy zone, which system comprises:
a source of a cell-manipulating factor;
a factor placement device connected to said factor source and configured for placing said factor in tissue in said therapy zone;
a force transducer configured for placement in said tissue and applying said factor to said tissue in said therapy zone;
a programmable controller connected to and controlling said factor placement device and said force transducer;
a non-dermal derived tissue scaffolding configured for implantation within in-vivo tissue in said therapy zone; and
wherein said tissue scaffolding is configured for enhancing cellular-level reactions within said therapy zone.

11. The medical cellular factor control system according to claim 10, which includes:
a sensor connected to said therapy zone and said controller;
wherein said sensor is configured for providing an input signal to said controller corresponding to an in vivo condition.

12. The medical cellular factor control system according to claim 11 wherein said in vivo condition detected by said sensor is chosen from among the group comprising: microbial activity; therapy zone healing status; blood pressure; pharmacological presence; lymphatic fluid presence; temperature; infection; antibiotic levels; and protein levels.

13. The medical cellular factor control system according to claim 10, which includes a cell manipulating factor chosen from the group comprising: physical contact, fluidic contact, fluid pressure gradient, pressure wave, osmolar, osmotic, oncotic, mechano/transductive, electromagnetic force (EMF), pharmacological, chemical, antimicrobial, fluidic, bioengineered cells for seeding, thermal, acoustic, and ultrasound.
